(19) United States
(12) Patent Application Publication
(10) Pub. No.: US 2025/0205217 A1
(43) Pub. Date: Jun. 26, 2025

(54) METHODS OF IMPROVING CANCER IMMUNOTHERAPY

(71) Applicants: THE REGENTS OF THE UNIVERSITY OF CALIFORNIA, Oakland, CA (US); BETH ISRAEL DEACONESS MEDICAL CENTER, INC., Boston, MA (US)

(72) Inventors: Dipak PANIGRAHY, Whitman, MA (US); Bruce D. HAMMOCK, Davis, CA (US); Sung Hee HWANG, Woodland, CA (US); Eva ROTHENBERGER, Brookline, MA (US)

(21) Appl. No.: 18/851,927

(22) PCT Filed: Mar. 31, 2023
(86) PCT No.: PCT/US2023/065240

§ 371 Date: Sep. 27, 2024

Related U.S. Application Data
(60) Provisional application No. 63/326,504, filed on Apr. 1, 2022.

(51) Int. Cl. A61K 31/4468; A23L 33/00; A61K 31/415; A61K 31/7068; A61K 33/243; A61K 39/00; A61P 35/00; C07K 16/28

(52) U.S. Cl. CPC A61K 31/4468; A23L 33/30; A61K 31/415; A61K 31/7068; A61K 33/243; A61P 35/00; C07K 16/2818; A61K 2039/505

(57) ABSTRACT

Provided herein are methods of preventing, suppressing, or treating cancer in a subject as well as methods of prolonging survival of a subject in need of cancer treatment. Such methods include administering (i) at least one immunotherapeutic agent, and (ii) a dual inhibitor of COX-2 and sEH; or (i) at least one immunotherapeutic agent, and (ii) an sEH inhibitor. The described methods further include optionally administering (iii) one or more chemotherapeutic agents, and/or (iv) a high omega-3 diet to the subject. Also provided herein are preventing, suppressing, or treating cancer in a subject as well as methods of prolonging survival of a subject in need of cancer treatment by administering (i) a high omega-3 diet or a high omega-6 diet, and optionally further including (ii) at least one immunotherapeutic agent.

### CROSS-REFERENCES TO RELATED APPLICATIONS

This is an application claiming priority benefit under 35 U.S.C. § 119 (e) of U.S. Provisional Application No. 63/326,504 filed Apr. 1, 2022, which is herein incorporated by reference in its entirety for all purposes.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Immunotherapy has become an increasingly available option for the treatment of various cancers. The positive benefits of immunotherapy are well documented; however, immunotherapy does not work in all contexts or with all patients. For example, immunotherapy does not work in 80% of bladder cancer patients and exhibits toxicity.

As such, there is a need in the art to identify additional treatment options for cancer patients. The present disclosure addresses these needs and provides related advantages as well.

### BRIEF SUMMARY OF THE INVENTION

Provided herein are methods of preventing, suppressing, or treating cancer in a subject, the method comprising administering to the subject (i) at least one immunotherapeutic agent, and (ii) a dual inhibitor of COX-2 and sEH.

In some aspects, provided herein are methods prolonging survival of a subject in need of cancer treatment, the method comprising administering to the subject (i) at least one immunotherapeutic agent, and (ii) a dual inhibitor of COX-2 and sEH.

Provided herein are methods of preventing, suppressing, or treating cancer in a subject, the method comprising administering to the subject (i) at least one immunotherapeutic agent, and (ii) a soluble epoxide hydrolase (sEH) inhibitor.

In some aspects, provided herein are methods prolonging survival of a subject in need of cancer treatment, the method comprising administering to the subject (i) at least one immunotherapeutic agent, and (ii) an sEH inhibitor.

In some embodiments, the dual inhibitor of COX-2 and sEH is a compound of Formula I

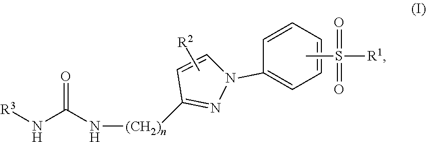

wherein the variable positions are as defined herein.

In other embodiments, the compound of Formula I is:

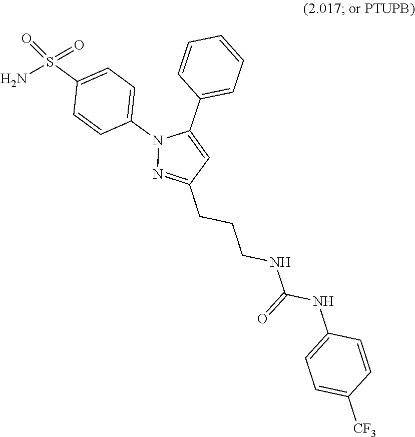 [SMILES: NS(=O)(=O)c1ccc(-n2nc(CCCNC(=O)Nc3ccc(C(F)(F)F)cc3)cc2-c2ccccc2)cc1]

or a pharmaceutically acceptable salt thereof.

In some embodiments, the sEH inhibitor is a compound of Formula II

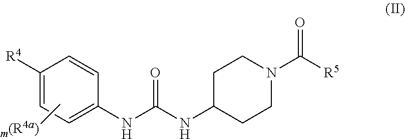

wherein the variable positions are as defined herein.

In some embodiments, the compound of Formula II has the structure

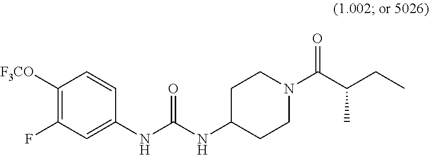 [SMILES: CC[C@H](C)C(=O)N1CCC(NC(=O)Nc2ccc(OC(F)(F)F)c(F)c2)CC1]

or a pharmaceutically acceptable salt thereof.

In some embodiments, the at least one immunotherapeutic agent is an immune checkpoint inhibitor(s) block(s) the activity of at least one of PD-1, PD-L1, LAG-3 or CTLA-4. In some embodiments, the at least one immunotherapeutic agent is an immune checkpoint inhibitor(s) block(s) the activity of at least one of PD-1, PD-L1, or CTLA-4.

In some embodiments, two immune checkpoint inhibitors are administered: a first immune checkpoint inhibitor blocking the activity of PD-1 or PD-L1, and a second immune checkpoint inhibitor blocking the activity of LAG-3 or CTLA-4. In some embodiments, one immune checkpoint inhibitor is administered, blocking the activity of PD-1 or PD-L1. In some embodiments, one immune checkpoint inhibitor is administered, blocking the activity of PD-1.

In some embodiments, one immune checkpoint inhibitor is administered, blocking the activity of LAG-3. In some embodiments, one immune checkpoint inhibitor is administered, blocking the activity of CTLA-4.

In some embodiments, the methods described herein further include administering (iii) one or more chemotherapeutic agents.

In some embodiments, the one or more chemotherapeutic agents are each independently selected from the group consisting of a platinum coordination complex and a nucleoside analog. In some embodiments, the platinum coordination complex is cisplatin and the nucleoside analogue is gemcitabine.

In some embodiments, the methods described herein further include administering (iv) a high omega-3 or a high omega-6 diet to the subject. In some embodiments, the subject is administered a high omega-3 diet. In some embodiments, the subject is administered a high omega-6 diet.

In some embodiments, the cancer is bladder, ovarian, cervical, breast, testicular, prostate, head and neck, oral, esophageal, gastric, lung, pancreatic, skin, leukemia, colon or colorectal cancer. In some embodiments, the cancer is bladder cancer.

In some aspects, provided herein are methods of preventing, suppressing, or treating cancer in a subject or prolonging survival of a subject comprising administering to the subject a high omega-3 diet or a high omega-6 diet and at least one immunotherapeutic agent.

In some aspects, provided herein are methods of preventing, suppressing, or treating cancer in a subject or prolonging survival of a subject comprising administering to the subject a high omega-3 diet or a high omega-6 diet. In some embodiments, the subject is administered a high omega-3 diet. In some embodiments, the subject is administered a high omega-6 diet.

### BRIEF DESCRIPTION OF THE DRAWINGS

FIGS. 1A-B: (A) Shows a diagram of tumor introduction into mice; (B) shows how dual COX-2/sEH inhibitor (PTUPB (Compound 2.017)) enhances immunotherapy in a murine bladder cancer model (MB49).

FIGS. 2A-C: (A) Plots tumor volume as a function of treatment days, showing how PTUPB (Compound 2.017) and immunotherapy (anti-PD1) in combination with chemotherapy induce tumor regression in murine bladder cancer model (MB49); (B) shows pictures of the surgically removed tumors, illustrating significant size differences after 16 days of treatment for the various groups tested; and (C) plots the tumor weight of each tumor show in B after 16 days of treatment.

FIG. 3: sEH inhibitor 5026 synergizes with immunotherapy (murine anti-PD1 antibody obtained from BioExcel) in high omega-6 diet in murine bladder cancer model (MB49).

FIG. 4: High Omega-3 Diet enhances immunotherapy (anti-PD1) in bladder cancer model (MB49).

FIGS. 5A-F: Immunotherapy can induce sEH and COX-2 expression in cancer models. (A) Expression of sEH in RM1 prostate tumor tissue. (B) Expression of sEH in MB49 bladder tumor tissue. (C) Expression of sEH in MB49 bladder tumor tissue. (D) Expression of COX-2 in RM1 prostate tumor tissue. (E) Expression of COX-2 in MB49 bladder tumor tissue. (F) Expression of COX-2 in MB49 bladder tumor tissue.

FIGS. 6A-6E Dietary omega-3 supplementation inhibits tumor growth in both fast-growing and slow-growing tumor models in mice. (A) Prostate cancer (RM1) tumor growth over time. (B) MB49 bladder cancer tumor volume over time. (C & D) Prostate cancer (Tramp C1) tumor volume (D) tumor weight at sacrifice. (E) Prostate cancer (Tramp C1) tumor take rate.

FIGS. 7A-D Dietary omega-3 supplementation enhances the anti-tumor activity of immunotherapy (murine anti-PD-1 antibody obtained from BioExcel). (A) MB49 bladder cancer tumor volume over time. (B) Prostate cancer (RM1) tumor volume over time. (C) B16F10 melanoma tumor volume over time. (D) Prostate tumor growth in Fat-1 transgenic mice. Tumor cells (50,000 RM1 prostate cancer cells) were injected subcutaneously.

FIGS. 8A-B sEH inhibitor synergizes with immunotherapy (murine antibodies obtained from BioExcel) to induce potent tumor inhibition in mice on an omega-6 rich diet. (A) MB49 bladder cancer tumor volume over time. (B) B16F10 melanoma tumor volume over time. B16F10 melanoma tumor volume over time.

FIGS. 9A-F Dual COX-2/sEH inhibition blocks tumor growth and enhances chemotherapy and immunotherapy in murine bladder cancer model. (A & B). Mice were inoculated subcutaneously with 1×10⁶ MB49 cells. (C) Mice were inoculated subcutaneously with 1×10⁶ MB49 cells. (D and E). Gene expression levels of EGF and VEGF-C in tumor tissue after 16 days of treatment. (F) Mice were inoculated orthotopically into the bladder wall with 5×10⁵ MB49 cells.

FIG. 10A-C Altered oxylipin profiles in mice treated with diets, immunotherapy, and sEH inhibitor. (A) Heatmap of LCMS/MS oxylipin analysis performed on murine MB49 bladder cancer tumor tissue. (B) Treatment with anti-PD1 increased levels of 7,8-DiHDPE, 17,18-DiHETE, and 15,16-DiHODE in MB49 bladder cancer tissue. (C) Treatment with anti-PD1 decreased levels of 15(16)-EpODE, 12(13)-EpOME, 17(18)-EpETE and 14(15)-EpETrE in MB49 bladder cancer tissue.

### DETAILED DESCRIPTION

### Definitions

Units, prefixes, and symbols are denoted in their Systeme International de Unites (SI) accepted form. Numeric ranges are inclusive of the numbers defining the range. The headings provided herein are not limitations of the various aspects or embodiments, which can be had by reference to the specification as a whole. Accordingly, the terms defined immediately below are more fully defined by reference to the specification in its entirety. Terms not defined herein have their ordinary meaning as understood by a person of skill in the art.

The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The term “alkyl”, by itself or as part of another substituent, means, unless otherwise stated, a straight or branched chain hydrocarbon group, having the number of carbon atoms designated (i.e. C₁₋₈ means one to eight carbons). Examples of alkyl groups include methyl, ethyl, n-propyl, isopropyl, n-butyl, t-butyl, isobutyl, sec-butyl, n-pentyl, n-hexyl, n-heptyl, n-octyl, and the like. The term “alkenyl” refers to an unsaturated alkyl group having one or more double bonds. Similarly, the term “alkynyl” refers to an unsaturated alkyl group having one or more triple bonds. Examples of alkenyl groups include vinyl, 2-propenyl, crotyl, 2-isopentenyl, 2-(butadienyl), 2,4-pentadienyl and 3-(1,4-pentadienyl). Examples of alkynyl groups include ethynyl, 1- and 3-propynyl, 3-butynyl, and the higher homologs and isomers. The term “cycloalkyl” refers to hydrocarbon rings having the indicated number of ring atoms (e.g., C₃₋₆cycloalkyl) and being fully saturated or having no more than one double bond between ring vertices. “Cycloalkyl” is also meant to refer to bicyclic and polycyclic hydrocarbon rings such as, for example, bicyclo[2.2.1]heptane, bicyclo[2.2.2]octane, etc. The bicyclic or polycyclic rings may be fused, bridged, spiro or a combination thereof. The term “heterocycloalkyl” or “heterocyclyl” refers to a cycloalkyl group having the indicated number of ring members, and which contain from one to three heteroatoms selected from N, O, and S, wherein the nitrogen and sulfur atoms are optionally oxidized, and the nitrogen atom(s) are optionally quaternized. The heterocycloalkyl may be a monocyclic, a bicyclic or a polycyclic ring system. The bicyclic or polycyclic rings may be fused, bridged, spiro or a combination thereof. It is understood that the recitation for C4-12 heterocyclyl, refers to a group having from 4 to 12 ring members where at least one of the ring members is a heteroatom. Non limiting examples of heterocycloalkyl groups include pyrrolidine, imidazolidine, pyrazolidine, butyrolactam, valerolactam, imidazolidinone, tetrazolone, hydantoin, dioxolane, phthalimide, piperidine, 1,4-dioxane, morpholine, thiomorpholine, thiomorpholine-S-oxide, thiomorpholine-S,S-oxide, piperazine, pyran, pyridone, 3-pyrroline, thiopyran, pyrone, tetrahydrofuran, tetrahydrothiophene, quinuclidine, and the like. A heterocycloalkyl group can be attached to the remainder of the molecule through a ring carbon or a heteroatom.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively. Additionally, for dialkylamino groups, the alkyl portions can be the same or different and can also be combined to form a 3-7 membered ring with the nitrogen atom to which each is attached. Accordingly, a group represented as —NR^{a}R^{b} is meant to include piperidinyl, pyrrolidinyl, morpholinyl, azetidinyl and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “C₁₋₄ haloalkyl” is mean to include trifluoromethyl, 2,2,2-trifluoroethyl, 4-chlorobutyl, 3-bromopropyl, and the like.

The term “hydroxyalkyl” or “alkyl-OH” refers to an alkyl group, as defined above, where at least one (and up to three) of the hydrogen atoms is replaced with a hydroxy group. As for the alkyl group, hydroxyalkyl groups can have any suitable number of carbon atoms, such as C₁₋₆. Exemplary hydroxyalkyl groups include, but are not limited to, hydroxymethyl, hydroxyethyl (where the hydroxy is in the 1- or 2-position), hydroxypropyl (where the hydroxy is in the 1-, 2- or 3-position), and 2,3-dihydroxypropyl.

The term “aryl” means, unless otherwise stated, an aromatic hydrocarbon group which can be a single ring or multiple rings (up to three rings) which are fused together or linked covalently. Non-limiting examples of aryl groups include phenyl, naphthyl and biphenyl. The term “heteroaryl” refers to aryl groups (or rings) that contain from one to five heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of heteroaryl groups include pyridyl, pyridazinyl, pyrazinyl, pyrimindinyl, triazinyl, quinolinyl, quinoxalinyl, quinazolinyl, cinnolinyl, phthalaziniyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl and the like.

As used herein, the term “heteroatom” is meant to include oxygen (O), nitrogen (N), sulfur(S), phosphorus (P) and silicon (Si).

The disclosure herein further relates to prodrugs and bioisosteres thereof. Suitable bioisosteres, for example, will include carboxylate replacements (phosphonic acids, phosphinic acids, sulfonic acids, sulfinic acids, and acidic heterocyclic groups such as tetrazoles). Suitable prodrugs will include those conventional groups known to hydrolyze and/or oxidize under physiological conditions to provide a compound of Formula I.

The terms “patient” and “subject” include primates (especially humans), domesticated companion animals (such as dogs, cats, horses, and the like) and livestock (such as cattle, pigs, sheep, and the like).

As used herein, the term “treating” or “treatment” encompasses both disease-modifying treatment and symptomatic treatment, cither of which may be prophylactic (i.e., before the onset of symptoms, in order to prevent, delay or reduce the severity of symptoms) or therapeutic (i.e., after the onset of symptoms, in order to reduce the severity and/or duration of symptoms).

“cis-Epoxyeicosatrienoic acids” (“EETs”) are biomediators synthesized by cytochrome P450 epoxygenases.

“Epoxide hydrolases” (“EH;” EC 3.3.2.3) are enzymes in the alpha beta hydrolase fold family that add water to 3-membered cyclic ethers termed epoxides.

“Soluble epoxide hydrolase” (“sEH”) is an epoxide hydrolase which in endothelial and smooth muscle cells converts EETs to dihydroxy derivatives called dihydroxyeicosatrienoic acids (“DHETs”). The cloning and sequence of the murine sEH is set forth in Grant et al., J. Biol. Chem. 268(23):17628-17633 (1993). The cloning, sequence, and accession numbers of the human sEH sequence are set forth in Beetham et al., Arch. Biochem. Biophys. 305(1):197-201 (1993). The evolution and nomenclature of the gene is discussed in Beetham et al., DNA Cell Biol. 14(1):61-71 (1995). Soluble epoxide hydrolase represents a single highly conserved gene product with over 90% homology between rodent and human (Arand et al., FEBS Lett., 338:251-256 (1994)). Unless otherwise specified, as used herein, the terms “soluble epoxide hydrolase” and “sEH” refer to human sEH.

Unless otherwise specified, as used herein, the term “sEH inhibitor” (also abbreviated as “sEHI”) refers to an inhibitor of human sEH. Preferably, the inhibitor does not also inhibit the activity of microsomal epoxide hydrolase by more than 25% at concentrations at which the inhibitor inhibits sEH by at least 50%, and more preferably docs not inhibit mEH by more than 10% at that concentration. For convenience of reference, unless otherwise required by context, the term “sEH inhibitor” as used herein encompasses prodrugs which are metabolized to active inhibitors of sEH. Further for convenience of reference, and except as otherwise required by context, reference herein to a compound as an inhibitor of sEH includes reference to derivatives of that compound (such as an ester of that compound) that retain activity as an sEH inhibitor.

Cytochrome P450 (“CYP450”) metabolism produces cis-epoxydocosapentaenoic acids (“EpDPEs”) and cis-epoxyeicosatetraenoic acids (“EpETEs”) from docosahexaenoic acid (“DHA”) and eicosapentaenoic acid (“EPA”), respectively. These epoxides are known endothelium-derived hyperpolarizing factors (“EDHFs”). These EDHFs, and others yet unidentified, are mediators released from vascular endothelial cells in response to acetylcholine and bradykinin, and are distinct from the NOS-(nitric oxide) and COX-derived (prostacyclin) vasodilators. Overall cytochrome P450 (CYP450) metabolism of polyunsaturated fatty acids produces epoxides, such as EETs. 14(15)-EpETE, for example, is derived via epoxidation of the 14,15-double bond of EPA and is the ω-3 homolog of 14(15)-EpETrE (“14(15)EET”) derived via epoxidation of the 14,15-double bond of arachidonic acid.

The term “therapeutically effective amount” refers to that amount of the compound being administered sufficient to prevent, mitigate, decrease, reverse the development of one or more of the symptoms of the disease, condition or disorder being treated.

The terms “sustained release” and “extended release” are used in their conventional sense to refer to a drug formulation that provides for gradual release of a drug over an extended period of time, for example, 12 hours or more, and that preferably, although not necessarily, results in substantially steady-state blood levels of a drug over an extended time period.

The terms “systemic administration” and “systemically administered” refer to a method of administering agent to a mammal so that the agent/cells is delivered to sites in the body, including the targeted site of pharmaceutical action, via the circulatory system. Systemic administration includes, but is not limited to, oral, intranasal, rectal and parenteral (i.e., other than through the alimentary tract, such as intramuscular, intravenous, intra-arterial, transdermal and subcutaneous) administration.

The phrase “cause to be administered” refers to the actions taken by a medical professional (e.g., a physician), or a person controlling medical care of a subject, that control and/or permit the administration of the agent(s)/compound(s)/cell(s) at issue to the subject. Causing to be administered can involve diagnosis and/or determination of an appropriate therapeutic or prophylactic regimen, and/or prescribing particular agent(s)/compounds/cell(s) for a subject. Such prescribing can include, for example, drafting a prescription form, annotating a medical record, and the like.

The terms “patient,” “subject” or “individual” interchangeably refers to a non-human mammal, including primates (e.g., macaque, pan troglodyte, pongo), a domesticated mammal (e.g., felines, canines), an agricultural mammal (e.g., bovine, ovine, porcine, equine) and a laboratory mammal or rodent (e.g., rattus, murine, lagomorpha, hamster).

### General

As described above, immunotherapy induces anti-tumor activity in only a subset of cancer patients, leaving many patients urgently needing new therapeutic approaches to make immunotherapy more effective.

The present disclosure demonstrates for the first time that immunotherapy in combination with a dual COX-2/sEH inhibitor or an sEH inhibitor alone enhances the anti-tumor activity of immunotherapy. This can also be achieved with subjects on a high omega-6 diet as well as with subject on a high omega-3 diet.

The present disclosure demonstrates for the first time that immunotherapy in combination with a high omega-6 diet as well as with subject on a high omega-3 diet enhances the anti-tumor activity of immunotherapy.

Without being bound to any particular theory, it is believed that inhibition of soluble epoxide hydrolase (sEH) or inhibition of sEH and COX-2 prevents cancer progression via resolution of inflammation, anti-angiogenesis, and modification of the tumor microenvironment (e.g., reducing inflammatory and proangiogenic cytokines).

### Methods of Preventing, Suppressing, or Treating Cancer in a Subject

Provided herein are methods of preventing, suppressing, or treating cancer in a subject, the method comprising administering to the subject (i) at least one immunotherapeutic agent, and (ii) a dual inhibitor of COX-2 and sEH.

In some aspects, provided herein are methods prolonging survival of a subject in need of cancer treatment, the method comprising administering to the subject (i) at least one immunotherapeutic agent, and (ii) a dual inhibitor of COX-2 and sEH.

In some embodiments, COX-2 inhibition can be provided from a separate pharmacological agent. Thus, also contemplated herein are embodiments where the dual inhibition of COX-2 and sEH are provided by two different agents: an inhibitor of COX-2 and an inhibitor of sEH.

Provided herein are methods of preventing, suppressing, or treating cancer in a subject, the method comprising administering to the subject (i) at least one immunotherapeutic agent, and (ii) an sEH inhibitor.

In some aspects, provided herein are methods prolonging survival of a subject in need of cancer treatment, the method comprising administering to the subject (i) at least one immunotherapeutic agent, and (ii) an sEH inhibitor.

Subjects who may benefit from these treatment methods are those who have a cancer. In some embodiments, the subject has a cancer that is refractory to one or more previous treatments. In some embodiments, the subject did not respond to immunotherapy alone. In some embodiments, the subject has a cancer that has relapsed after an initial response to immunotherapy.

Provided herein are methods of preventing, suppressing, or treating cancer in a subject, the method comprising administering to the subject (i) a high omega-3 diet or a high omega-6 diet. In some embodiments, the subject is administered a high omega-3 diet. In some embodiments, the subject is administered a high omega-6 diet. In some embodiments, a subject is also administered (ii) at least one immunotherapeutic agent.

In some aspects, provided herein are methods prolonging survival of a subject in need of cancer treatment, the method comprising administering to the subject (i) a high omega-3 diet or a high omega-6 diet. In some embodiments, the subject is administered a high omega-3 diet. In some embodiments, the subject is administered a high omega-6 diet.

In some embodiments, the cancer is lymphoma, lung cancer, breast cancer, ovarian cancer, cervical cancer, prostate cancer, testicular cancer, gastric and intestinal cancers (including colon cancer and rectal cancer), hepatic cancer, oral cancer, esophageal cancer, bladder cancer, renal cancer, bladder cancer, or head and neck cancers. In some embodiments, the cancer is bladder, ovarian, cervical, breast, testicular, prostate, head and neck, oral, esophageal, gastric, lung, pancreatic, skin, leukemia, colon or colorectal cancer. In some embodiments, the subject has bladder cancer.

In some embodiments, the cancer is melanoma, non-small cell lung cancer (NSCLC), renal cell carcinoma, Hodgkin's lymphoma, head and neck squamous cell carcinoma (HNSCC), merkel cell carcinoma, a microsatellite instability-high (MSI-H) cancer, colorectal cancer, gastric cancer, hepatocellular carcinoma, cervical cancer, primary mediastinal large B-cell lymphoma (PMBCL), small cell lung cancer (SCLC), cutaneous squamous-cell carcinoma, basal cell carcinoma, bladder cancer, breast cancer, mismatch repair deficient (dMMR) cancer, Endometrial Carcinoma, Esophagus cancer, Malignant Pleural Mesothelioma, high tumor mutation burden (TMB-H) cancers. In some embodiments, the cancer is melanoma.

In some embodiments, the subject has a hematologic malignancy, which include without limitation lymphomas (such as but not limited to, non-Hodgkin's lymphoma, including Burkitt's lymphoma, and Hodgkin's lymphoma, as well as all subtypes associated with each), acute lymphocytic leukemia (ALL), chronic lymphocytic leukemia (CLL), acute myeloid leukemia (AML), chronic myeloid leukemia (CML), and adult T-cell leukemia lymphoma.

In some embodiments, the subject has a lung cancer. Exemplary lung cancers include without limitation adenocarcinoma, squamous carcinoma, bronchial carcinoma, bronchoalveolar carcinoma, large cell carcinoma, small-cell carcinoma, non-small cell lung carcinoma and metastatic lung cancer refractory to conventional chemotherapy.

In some embodiments, the subject has a hematologic malignancy such as multiple myeloma or plasmocytoma.

In some embodiments, the subject has a sarcoma. In some embodiments the sarcoma includes without limitation rhabdomyosarcoma, osteosarcoma, chondrosarcoma, myosarcoma, liposarcoma, fibrosarcoma and Ewing's sarcoma.

In some embodiments, the subject has a gastric, digestive and intestinal cancer, which include without limitation intestinal carcinoma, rectal carcinoma, colon carcinoma, familial adenomatous polyposis carcinoma, hereditary non-polyposis colorectal cancer, gastric carcinoma, craniopharyngioma, gall bladder carcinoma, esophageal carcinoma, pancreatic carcinoma and adenocarcinoma (including adenocarcinomas of the esophagus and stomach).

In some embodiments, the subject has a cancer of the head and neck. Cancers of the head and neck include without limitation larynx carcinoma, hypopharynx carcinoma, tongue carcinoma and salivary gland carcinoma.

In some embodiments, the subject has a urogenital cancers, which include without limitation labial carcinoma, ovarian carcinoma, cervix carcinoma, uterine corpus carcinoma, endometrium carcinoma, chorion carcinoma, prostate carcinoma, testis carcinoma, seminoma, urinary carcinoma, kidney carcinoma, renal carcinoma, and adenocarcinoma (including adenocarcinomas of the vagina, cervix, prostate, and urachus).

In some embodiments, the subject has a nervous and sensory system cancer, which include without limitation neuroblastoma, brain tumors, meningioma, ependymoma, medulloblastoma, peripheral neuroectodermal tumors, glioblastoma, astrocytoma, oligodendroglioma and retinoblastoma.

In some embodiments, the subject has a endocrine and glandular tissue cancer, which include without limitation pancreatic carcinoma, medullary thyroid carcinoma, follicular thyroid carcinoma, anaplastic thyroid carcinoma, papillary thyroid carcinoma, pheochromocytoma, adrenal tumors and adenocarcinoma.

In some embodiments, the subject has a hepatic cancers, which include without limitation hepatocellular carcinoma.

In some embodiments, the subject has a skin cancer, which include without limitation melanoma, basal cell carcinoma, squamous cell carcinoma and choroids melanoma.

In some embodiments, the subject has a cancer selected from: bladder cancer, prostate cancer, pancreatic cancer, and melanoma. In some embodiments, the subject has bladder cancer. In some embodiments, the subject has prostate cancer. In some embodiments, the subject has pancreatic cancer. In some embodiments, the subject has melanoma.

### Dual Inhibitors of COX-2 and sEH

Dual inhibitors of COX-2 and sEH have been previously disclosed. Each of these compounds are intended to be within the scope of the current disclosure.

In some embodiments, the dual inhibitor of COX-2 and sEH is a compound of Formula I

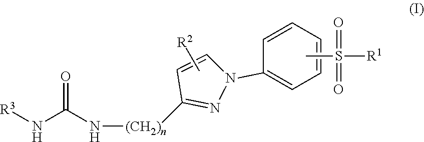

wherein
R¹ is selected from the group consisting of C₁₋₆ alkyl, —NR^{1a}R^{1b} and C₃₋₆ cycloalkyl; R^{1a} and R^{1b} are each independently selected from the group consisting of H and C₁₋₆ alkyl;
R² is selected from the group consisting of C₁₋₆ alkyl, C₃₋₆ cycloalkyl and aryl, wherein the cycloalkyl and aryl are each optionally substituted with C₁₋₆ alkyl;
R³ is selected from the group consisting of C₅₋₁₀ cycloalkyl and aryl, each optionally substituted with from 1 to 3 R^{3a} groups wherein each R^{3a} is independently selected from the group consisting of C₁₋₆ alkyl, C₁₋₆ alkoxy, halogen, C₁₋₆ haloalkyl and C₁₋₆ haloalkoxy;
subscript n is an integer from 0 to 6;
   and salts and isomers thereof.

In some embodiments, the compound of Formula I is represented by Formula Ia:

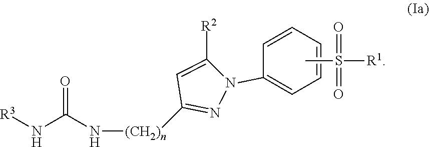

In some embodiments, the compound of Formula I is represented by Formula Ib:

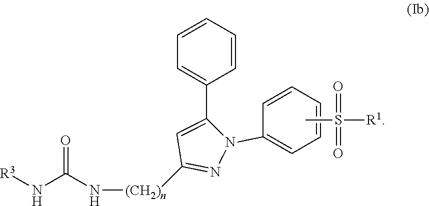

In some embodiments, the compound of Formula I is represented by Formula Ic:

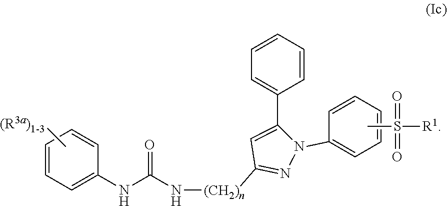

In some embodiments, the present invention provides a compound of Formula I, wherein R¹ is C₁₋₆ alkyl or —NR^{1a}R^{1b}; R^{1a} and R^{1b} are each independently H or C₁₋₆ alkyl; R² is aryl, optionally substituted with C₁₋₆ alkyl; and R³ is cycloalkyl or aryl, each optionally substituted with from 1 to 3 R^{3a} groups wherein each R^{3a} is independently C₁₋₆ alkyl, halogen, C₁₋₆ haloalkyl and C₁₋₆ haloalkoxy. In some other embodiments, R¹ is methyl, ethyl, propyl, —NH2 and —NMe₂; R² is phenyl, optionally substituted with a member selected from methyl, ethyl or propyl; and R³ is selected from cyclohexyl, cycloheptyl, cyclooctyl, adamantyl or phenyl, wherein the phenyl is optionally substituted with from 1 to 3 R^{3a} groups wherein each R^{3a} is independently methyl, ethyl, propyl, Cl, Br, I, —CF₃ or —OCF₃.

In some other embodiments, the present invention provides a compound of Formula I, selected from those in Table 1.

TABLE 1
Compounds of Formula I
Structure

| | |
|---|---|
| 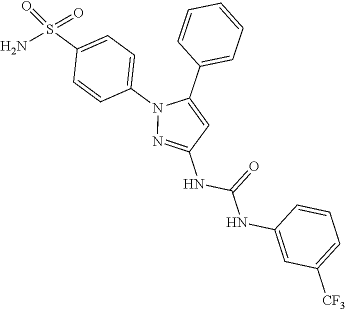 [SMILES: NS(=O)(=O)c1ccc(-n2nc(NC(=O)Nc3cccc(C(F)(F)F)c3)cc2-c2ccccc2)cc1] | 2.001 |
| 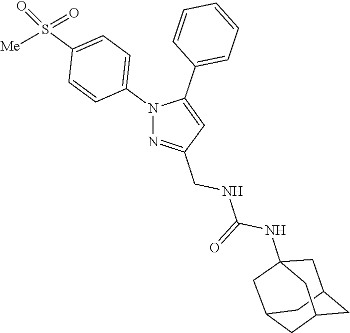 [SMILES: CS(=O)(=O)c1ccc(-n2nc(CNC(=O)NC34CC5CC(CC(C5)C3)C4)cc2-c2ccccc2)cc1] | 2.002 |
| 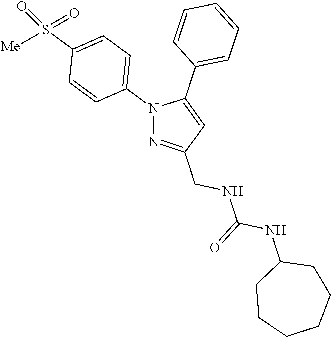 [SMILES: CS(=O)(=O)c1ccc(-n2nc(CNC(=O)NC3CCCCCC3)cc2-c2ccccc2)cc1] | 2.003 |
| 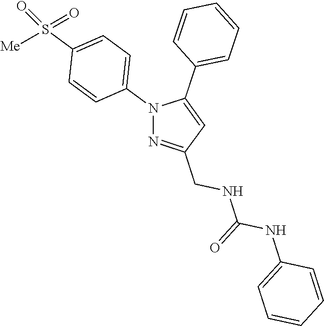 [SMILES: CS(=O)(=O)c1ccc(-n2nc(CNC(=O)Nc3ccccc3)cc2-c2ccccc2)cc1] | 2.004 |
| 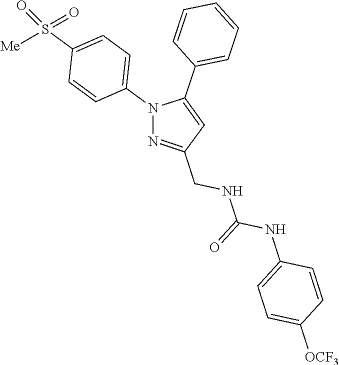 [SMILES: CS(=O)(=O)c1ccc(-n2nc(CNC(=O)Nc3ccc(OC(F)(F)F)cc3)cc2-c2ccccc2)cc1] | 2.005 |
| 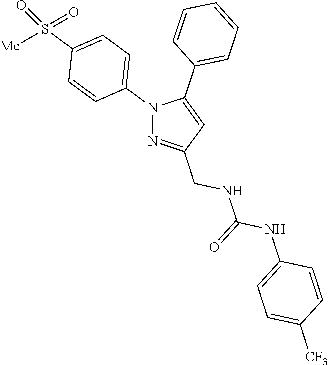 [SMILES: CS(=O)(=O)c1ccc(-n2nc(CNC(=O)Nc3ccc(C(F)(F)F)cc3)cc2-c2ccccc2)cc1] | 2.006 |
| 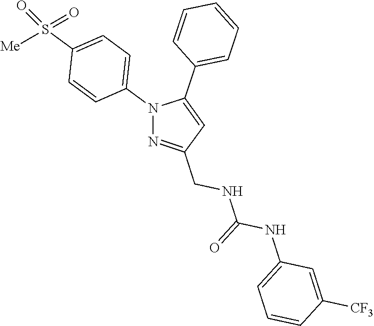 [SMILES: CS(=O)(=O)c1ccc(-n2nc(CNC(=O)Nc3cccc(C(F)(F)F)c3)cc2-c2ccccc2)cc1] | 2.007 |
| 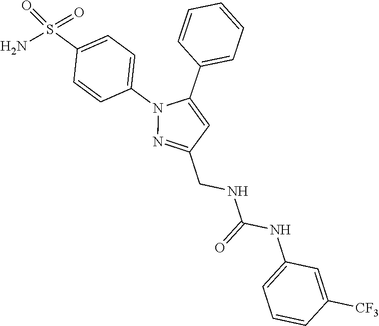 [SMILES: NS(=O)(=O)c1ccc(-n2nc(CNC(=O)Nc3cccc(C(F)(F)F)c3)cc2-c2ccccc2)cc1] | 2.008 |
| 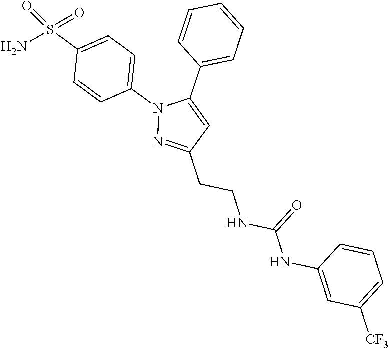 [SMILES: NS(=O)(=O)c1ccc(-n2nc(CCNC(=O)Nc3cccc(C(F)(F)F)c3)cc2-c2ccccc2)cc1] | 2.009 |
| 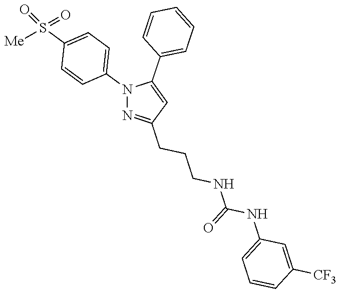 [SMILES: CS(=O)(=O)c1ccc(-n2nc(CCCNC(=O)Nc3cccc(C(F)(F)F)c3)cc2-c2ccccc2)cc1] | 2.010 |
| 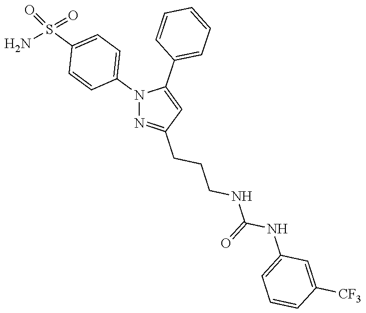 [SMILES: NS(=O)(=O)c1ccc(-n2nc(CCCNC(=O)Nc3cccc(C(F)(F)F)c3)cc2-c2ccccc2)cc1] | 2.011 |
| 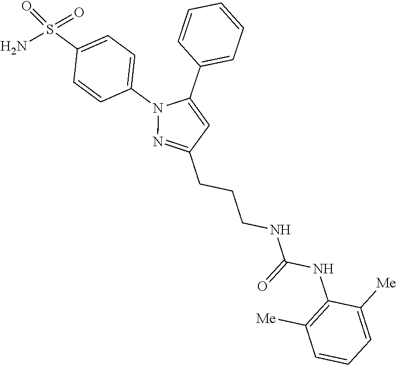 [SMILES: Cc1cccc(C)c1NC(=O)NCCCc1cc(-c2ccccc2)n(-c2ccc(S(N)(=O)=O)cc2)n1] | 2.012 |
| 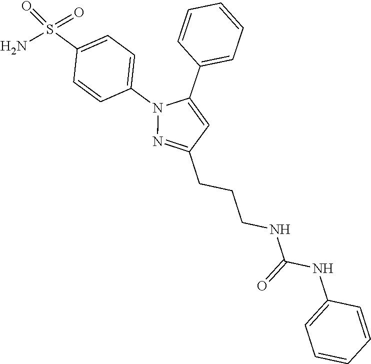 [SMILES: NS(=O)(=O)c1ccc(-n2nc(CCCNC(=O)Nc3ccccc3)cc2-c2ccccc2)cc1] | 2.013 |
| 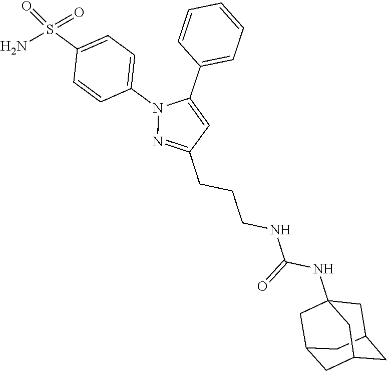 [SMILES: NS(=O)(=O)c1ccc(-n2nc(CCCNC(=O)NC34CC5CC(CC(C5)C3)C4)cc2-c2ccccc2)cc1] | 2.014 |
| 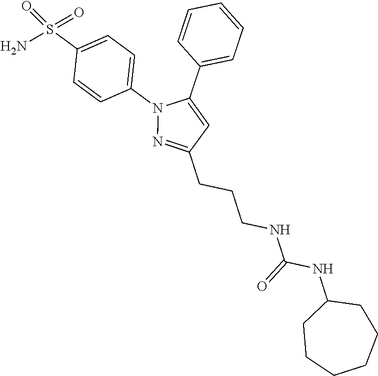 [SMILES: NS(=O)(=O)c1ccc(-n2nc(CCCNC(=O)NC3CCCCCC3)cc2-c2ccccc2)cc1] | 2.015 |
| 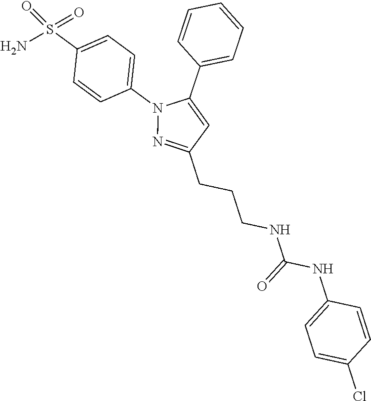 [SMILES: NS(=O)(=O)c1ccc(-n2nc(CCCNC(=O)Nc3ccc(Cl)cc3)cc2-c2ccccc2)cc1] | 2.016 |
| 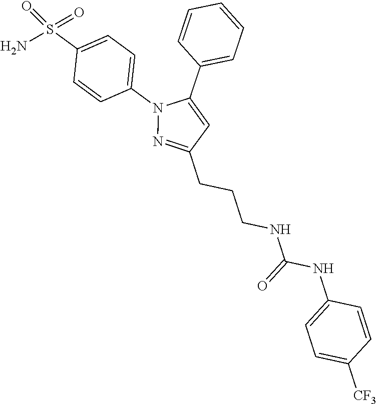 [SMILES: NS(=O)(=O)c1ccc(-n2nc(CCCNC(=O)Nc3ccc(C(F)(F)F)cc3)cc2-c2ccccc2)cc1] | 2.017 |
| 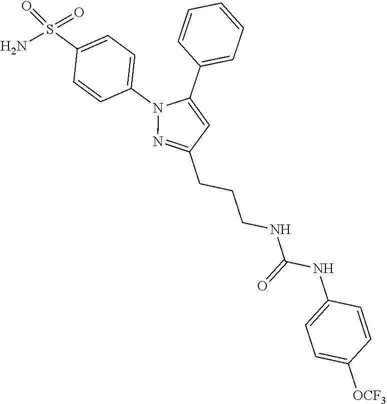 [SMILES: NS(=O)(=O)c1ccc(-n2nc(CCCNC(=O)Nc3ccc(OC(F)(F)F)cc3)cc2-c2ccccc2)cc1] | 2.018 |
| 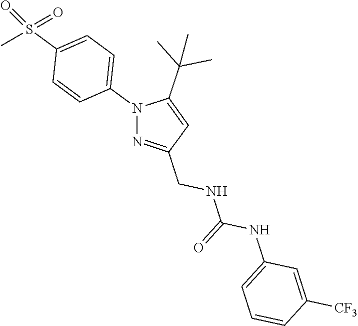 [SMILES: CC(C)(C)c1cc(CNC(=O)Nc2cccc(C(F)(F)F)c2)nn1-c1ccc(S(C)(=O)=O)cc1] | 2.019 |
| 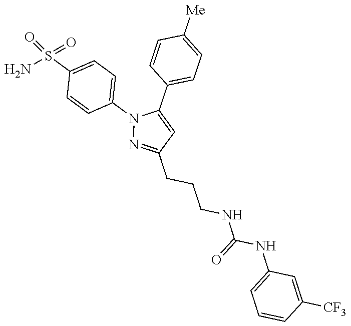 [SMILES: Cc1ccc(-c2cc(CCCNC(=O)Nc3cccc(C(F)(F)F)c3)nn2-c2ccc(S(N)(=O)=O)cc2)cc1] | 2.020 |

In other embodiments, the compound can be:

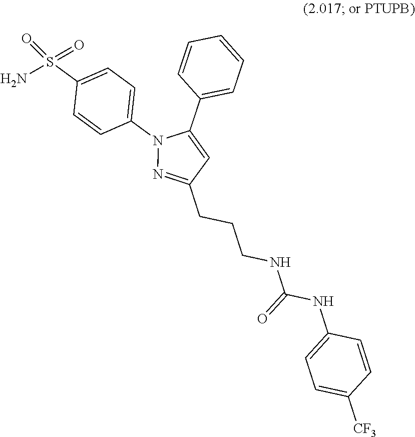 [SMILES: NS(=O)(=O)c1ccc(-n2nc(CCCNC(=O)Nc3ccc(C(F)(F)F)cc3)cc2-c2ccccc2)cc1]

or a pharmaceutically acceptable salt thereof.

In other embodiments, the compound can be:

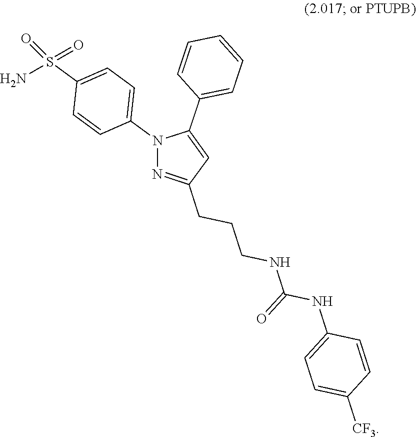 [SMILES: NS(=O)(=O)c1ccc(-n2nc(CCCNC(=O)Nc3ccc(C(F)(F)F)cc3)cc2-c2ccccc2)cc1]

The compounds of Formula I may exist as salts. The present invention includes such salts. Typically, the salts used are pharmaceutically acceptable salts.

Pharmaceutically acceptable salts include salts of the active compounds which are prepared with relatively nontoxic acids or bases, depending on the particular substituents found on the compounds described herein. When compounds of Formula I contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of Formula I contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of Formula I contain both basic and acidic functionalities that allow the compounds to be converted into cither base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Certain compounds of Formula I can exist in unsolvated forms as well as solvated forms, including hydrated forms. In general, the solvated forms are equivalent to unsolvated forms and are encompassed within the scope of the present invention. Certain compounds of Formula I may exist in multiple crystalline or amorphous forms. In general, all physical forms are equivalent for the uses contemplated by the present invention and are intended to be within the scope of the present invention.

Certain compounds of Formula I possess asymmetric carbon atoms (optical centers) or double bonds; the enantiomers, racemates, diastereomers, tautomers, geometric isomers, stereoisomeric forms that may be defined, in terms of absolute stereochemistry, as (R)- or (S)- or, as (D)- or (L)- for amino acids, and individual isomers are encompassed within the scope of the present invention. The compounds of Formula I do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques.

Isomers include compounds having the same number and kind of atoms, and hence the same molecular weight, but differing in respect to the structural arrangement or configuration of the atoms.

It will be apparent to one skilled in the art that certain compounds of this invention may exist in tautomeric forms, all such tautomeric forms of the compounds being within the scope of the invention. Tautomer includes one of two or more structural isomers which exist in equilibrium and which are readily converted from one isomeric form to another.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, the compounds of Formula I may also contain unnatural proportions of atomic isotopes at one or more of the atoms that constitute such compounds. For example, the compounds of Formula I may be radiolabeled with radioactive isotopes, such as for example deuterium (²H), tritium (³H), iodine-125 (¹²⁵I), carbon-13 (¹³C), or carbon-14 (¹⁴C). All isotopic variations of the compounds of Formula I, whether radioactive or not, are encompassed within the scope of the present invention.

In addition to salt forms, the compounds of Formula I can be prepared as prodrugs. Prodrugs of the compounds described herein are those compounds that readily undergo chemical changes under physiological conditions to provide the compounds of Formula I. Additionally, prodrugs can be converted to the compounds of Formula I by chemical or biochemical methods in an ex vivo environment. For example, prodrugs can be slowly converted to the compounds of Formula I when placed in a transdermal patch reservoir with a suitable enzyme or chemical reagent.

The compounds of Formula I can be made by a variety of methods known in the art.

### Inhibitors of COX-2

Commercially available NSAIDs that find use in the methods and compositions of the invention include the traditional NSAIDs diclofenac potassium, diclofenac sodium, diclofenac sodium with misoprostol, diflunisal, etodolac, fenoprofen calcium, flurbiprofen, ibuprofen, indomethacin, ketoprofen, meclofenamate sodium, mefenamic acid, meloxicam, nabumetone, naproxen sodium, piroxicam, tolmetin sodium, the selective COX-2 inhibitors celecoxib, rofecoxib, and valdecoxib, the acetylated salicylates, such as aspirin, and the non-acetylated salicylates, such as magnesium salicylate, choline salicylate, salsalate, salicylic acid esters and sodium salicylate. In some embodiments, the preferential or selective inhibitor of COX-2 is selected from the group consisting of celecoxib, valdecoxib, lumiracoxib, etoricoxib, parecoxib, rofecoxib, nabumetone, meloxicam, and mixtures thereof.

### Inhibitors of sEH (sEH Inhibitors)

Inhibitors of sEH have been previously disclosed. Each of these compounds are intended to be within the scope of the current disclosure.

In some embodiments, the sEH inhibitor is a compound of Formula II

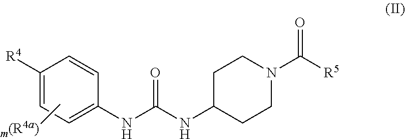

wherein
R⁴ is —OCF₃ or —CF₃;
each R^{4a} is selected from the group consisting of H, halogen, C₁₋₆ alkyl, C₁₋₆ alkoxy, C₁₋₆ haloalkyl, C₁₋₆ haloalkoxy, —O-aryl, 5- to 6-membered heterocycloalkyl having 1 to 3 heteroatoms as ring vertices selected from N, O, and S, —OH, —NO₂, and —C(O)OR^{4b};
R⁵ is selected from the group consisting of C₁₋₆ alkyl, C₁₋₆ haloalkyl, C₁₋₆ hydroxyalkyl, C₁₋₆ alkoxy, C₁₋₆ haloalkoxy, —X⁵—C₃₋₆ cycloalkyl, —X⁵-3- to 6-membered heterocycloalkyl having 1 to 3 heteroatoms as ring vertices selected from N, O, and S, and —X⁵-5- to 6-membered heteroaryl having 1 to 3 heteroatom as ring vertices selected from N, O, and S, wherein
R⁵ is optionally substituted with from 1 to 3 substituents selected from the group consisting of C₁₋₄ alkyl, C₁₋₄ haloalkyl, hydroxyl, —C(O)OR^{5a}, and —C₁₋₄-alkylene-C(O)OR^{5a};
X⁵ is selected from a bond and C₁₋₃ alkylene
R^{4b} and R^{5a} are each independently H or C₁₋₆ alkyl; and
subscript m is an integer from 0 to 2.

In some embodiments, the compound of Formula II is represented by Formula IIa

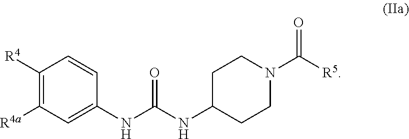

In some embodiments, R⁴ in Formula II or IIa is —OCF₃.

In some embodiments, R⁴ in Formula II or IIa is —CF₃.

In some embodiments, m is 1 and R^{4a} in Formula II or IIa is selected from the group consisting of —CF₃, Cl, Br, F, and —OCF₃.

In some embodiments, m is 1 and R^{4a} in Formula II or IIa is F.

In some embodiments, R⁵ in Formula II or IIa is selected from the group consisting of C₁₋₆ alkyl, C₁₋₆ haloalkyl, C₁₋₆ hydroxyalkyl, C₁₋₆ alkoxy, C₁₋₆ haloalkoxy, C₃₋₆ cycloalkyl, and 3- to 6-membered heterocycloalkyl having 1 to 3 heteroatoms as ring vertices selected from N, O, and S, and -5- to 6-membered heteroaryl having 1 to 3 heteroatom as ring vertices selected from N, O, and S.

In some embodiments, R⁵ in Formula II or IIa is selected from the group consisting of C₁₋₆ alkyl, C₁₋₆ haloalkyl and C₁₋₆ hydroxyalkyl.

In some embodiments, compounds of Formula II are selected from a compound in Table 2.

TABLE 2
Compounds of Formula II
Structure

| | |
|---|---|
| 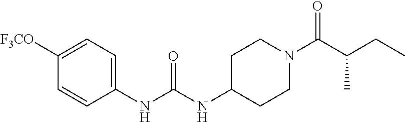 [SMILES: CC[C@H](C)C(=O)N1CCC(NC(=O)Nc2ccc(OC(F)(F)F)cc2)CC1] | 1.001 |
| 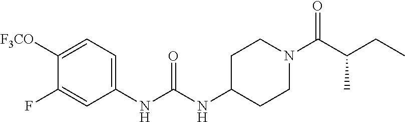 [SMILES: CC[C@H](C)C(=O)N1CCC(NC(=O)Nc2ccc(OC(F)(F)F)c(F)c2)CC1] | 1.002 |
| 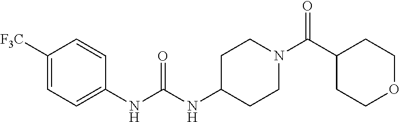 [SMILES: O=C(Nc1ccc(C(F)(F)F)cc1)NC1CCN(C(=O)C2CCOCC2)CC1] | 1.003 |
| 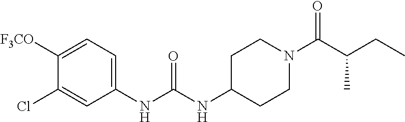 [SMILES: CC[C@H](C)C(=O)N1CCC(NC(=O)Nc2ccc(OC(F)(F)F)c(Cl)c2)CC1] | 1.004 |
| 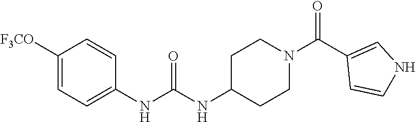 [SMILES: O=C(Nc1ccc(OC(F)(F)F)cc1)NC1CCN(C(=O)c2cc[nH]c2)CC1] | 1.005 |
| 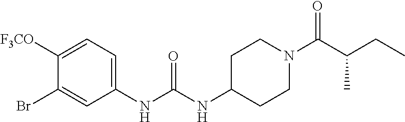 [SMILES: CC[C@H](C)C(=O)N1CCC(NC(=O)Nc2ccc(OC(F)(F)F)c(Br)c2)CC1] | 1.006 |
| 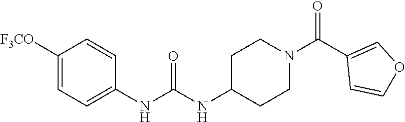 [SMILES: O=C(Nc1ccc(OC(F)(F)F)cc1)NC1CCN(C(=O)c2ccoc2)CC1] | 1.007 |
| 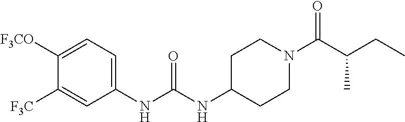 [SMILES: CC[C@H](C)C(=O)N1CCC(NC(=O)Nc2ccc(OC(F)(F)F)c(C(F)(F)F)c2)CC1] | 1.008 |
| 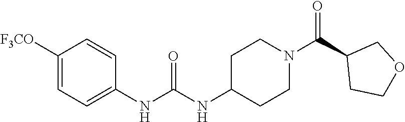 [SMILES: O=C(Nc1ccc(OC(F)(F)F)cc1)NC1CCN(C(=O)[C@@H]2CCOC2)CC1] | 1.009 |
| 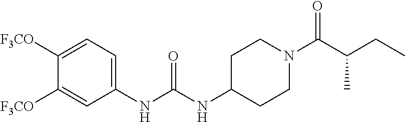 [SMILES: CC[C@H](C)C(=O)N1CCC(NC(=O)Nc2ccc(OC(F)(F)F)c(OC(F)(F)F)c2)CC1] | 1.010 |
| 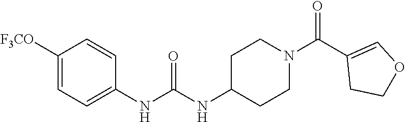 [SMILES: O=C(Nc1ccc(OC(F)(F)F)cc1)NC1CCN(C(=O)C2=COCC2)CC1] | 1.011 |
| 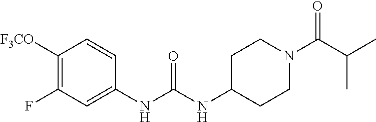 [SMILES: CC(C)C(=O)N1CCC(NC(=O)Nc2ccc(OC(F)(F)F)c(F)c2)CC1] | 1.012 |
| 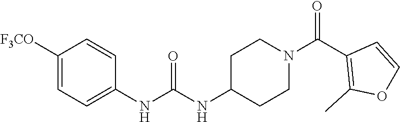 [SMILES: Cc1occc1C(=O)N1CCC(NC(=O)Nc2ccc(OC(F)(F)F)cc2)CC1] | 1.013 |
| 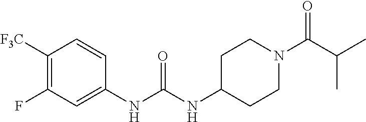 [SMILES: CC(C)C(=O)N1CCC(NC(=O)Nc2ccc(C(F)(F)F)c(F)c2)CC1] | 1.014 |
| 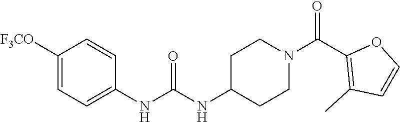 [SMILES: Cc1ccoc1C(=O)N1CCC(NC(=O)Nc2ccc(OC(F)(F)F)cc2)CC1] | 1.015 |
| 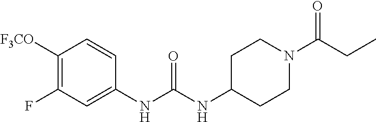 [SMILES: CCC(=O)N1CCC(NC(=O)Nc2ccc(OC(F)(F)F)c(F)c2)CC1] | 1.016 |
| 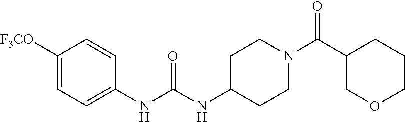 [SMILES: O=C(Nc1ccc(OC(F)(F)F)cc1)NC1CCN(C(=O)C2CCCOC2)CC1] | 1.017 |
| 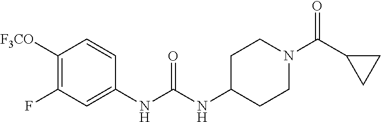 [SMILES: O=C(Nc1ccc(OC(F)(F)F)c(F)c1)NC1CCN(C(=O)C2CC2)CC1] | 1.018 |
| 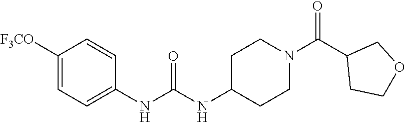 [SMILES: O=C(Nc1ccc(OC(F)(F)F)cc1)NC1CCN(C(=O)C2CCOC2)CC1] | 1.019 |
| 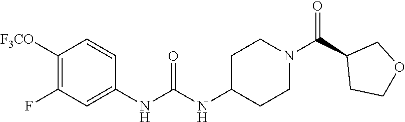 [SMILES: O=C(Nc1ccc(OC(F)(F)F)c(F)c1)NC1CCN(C(=O)[C@@H]2CCOC2)CC1] | 1.020 |
| 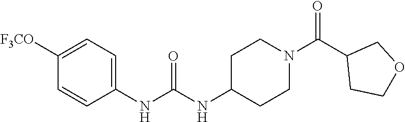 [SMILES: O=C(Nc1ccc(OC(F)(F)F)cc1)NC1CCN(C(=O)C2CCOC2)CC1] | 1.021 |
| 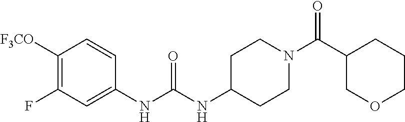 [SMILES: O=C(Nc1ccc(OC(F)(F)F)c(F)c1)NC1CCN(C(=O)C2CCCOC2)CC1] | 1.022 |
| 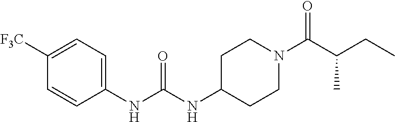 [SMILES: CC[C@H](C)C(=O)N1CCC(NC(=O)Nc2ccc(C(F)(F)F)cc2)CC1] | 1.023 |
| 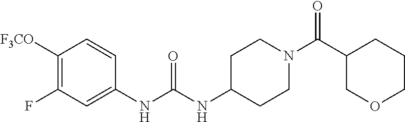 [SMILES: O=C(Nc1ccc(OC(F)(F)F)c(F)c1)NC1CCN(C(=O)C2CCCOC2)CC1] | 1.024 |
| 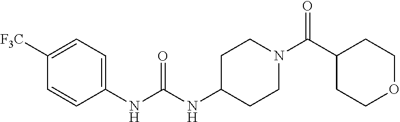 [SMILES: O=C(Nc1ccc(C(F)(F)F)cc1)NC1CCN(C(=O)C2CCOCC2)CC1] | 1.025 |
| 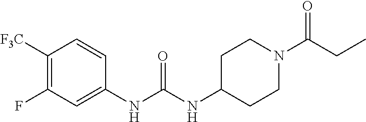 [SMILES: CCC(=O)N1CCC(NC(=O)Nc2ccc(C(F)(F)F)c(F)c2)CC1] | 1.026 |
| 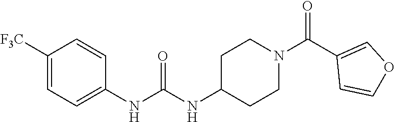 [SMILES: O=C(Nc1ccc(C(F)(F)F)cc1)NC1CCN(C(=O)c2ccoc2)CC1] | 1.027 |
| 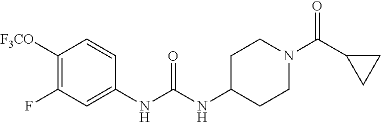 [SMILES: O=C(Nc1ccc(OC(F)(F)F)c(F)c1)NC1CCN(C(=O)C2CC2)CC1] | 1.028 |
| 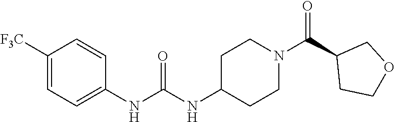 [SMILES: O=C(Nc1ccc(C(F)(F)F)cc1)NC1CCN(C(=O)[C@@H]2CCOC2)CC1] | 1.029 |
| 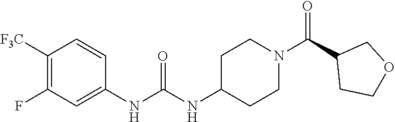 | 1.030 |
| 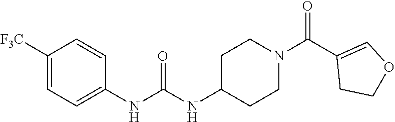 [SMILES: O=C(Nc1ccc(C(F)(F)F)cc1)NC1CCN(C(=O)C2=COCC2)CC1] | 1.031 |
| 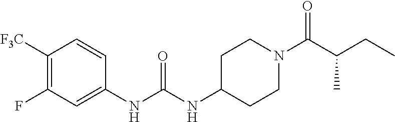 [SMILES: CC[C@H](C)C(=O)N1CCC(NC(=O)Nc2ccc(C(F)(F)F)c(F)c2)CC1] | 1.032 |
| 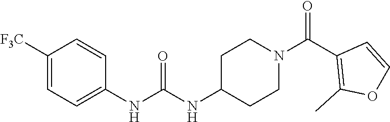 [SMILES: Cc1occc1C(=O)N1CCC(NC(=O)Nc2ccc(C(F)(F)F)cc2)CC1] | 1.033 |
| 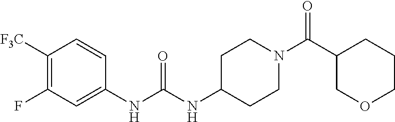 [SMILES: O=C(Nc1ccc(C(F)(F)F)c(F)c1)NC1CCN(C(=O)C2CCCOC2)CC1] | 1.034 |
| 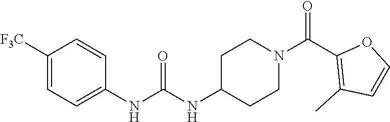 [SMILES: Cc1ccoc1C(=O)N1CCC(NC(=O)Nc2ccc(C(F)(F)F)cc2)CC1] | 1.035 |
| 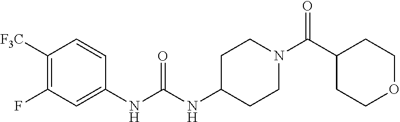 [SMILES: O=C(Nc1ccc(C(F)(F)F)c(F)c1)NC1CCN(C(=O)C2CCOCC2)CC1] | 1.036 |
| 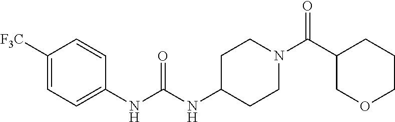 [SMILES: O=C(Nc1ccc(C(F)(F)F)cc1)NC1CCN(C(=O)C2CCCOC2)CC1] | 1.037 |
| 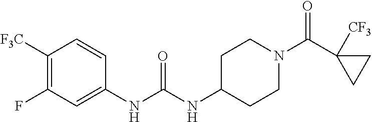 [SMILES: O=C(Nc1ccc(C(F)(F)F)c(F)c1)NC1CCN(C(=O)C2(C(F)(F)F)CC2)CC1] | 1.038 |
| 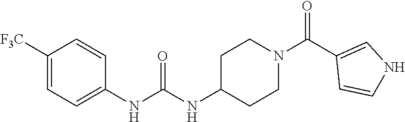 [SMILES: O=C(Nc1ccc(C(F)(F)F)cc1)NC1CCN(C(=O)c2cc[nH]c2)CC1] | 1.039 |
| 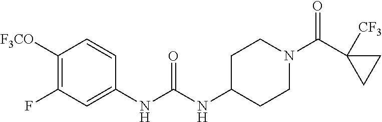 [SMILES: O=C(Nc1ccc(OC(F)(F)F)c(F)c1)NC1CCN(C(=O)C2(C(F)(F)F)CC2)CC1] | 1.040 |
| 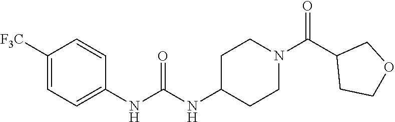 [SMILES: O=C(Nc1ccc(C(F)(F)F)cc1)NC1CCN(C(=O)C2CCOC2)CC1] | 1.041 |
| 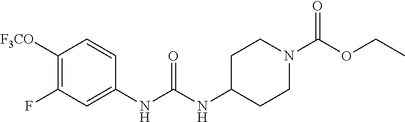 [SMILES: CCOC(=O)N1CCC(NC(=O)Nc2ccc(OC(F)(F)F)c(F)c2)CC1] | 1.042 |
| 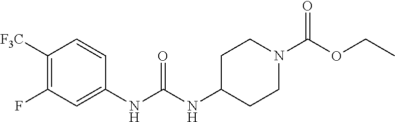 [SMILES: CCOC(=O)N1CCC(NC(=O)Nc2ccc(C(F)(F)F)c(F)c2)CC1] | 1.043 |
| 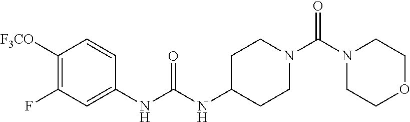 [SMILES: O=C(Nc1ccc(OC(F)(F)F)c(F)c1)NC1CCN(C(=O)N2CCOCC2)CC1] | 1.044 |
| 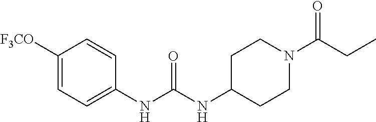 [SMILES: CCC(=O)N1CCC(NC(=O)Nc2ccc(OC(F)(F)F)cc2)CC1] | 1.045 |

In some embodiments, the compound of Formula II has the structure

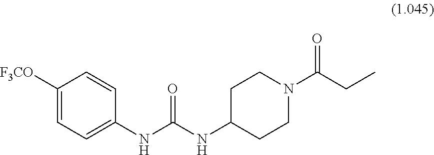 [SMILES: CCC(=O)N1CCC(NC(=O)Nc2ccc(OC(F)(F)F)cc2)CC1]

or a pharmaceutically acceptable salt thereof.

In some embodiments, the compound of Formula II has the structure

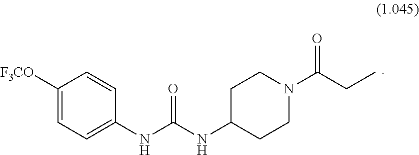 [SMILES: CCC(=O)N1CCC(NC(=O)Nc2ccc(OC(F)(F)F)cc2)CC1]

In some embodiments, the compound of Formula II has the structure

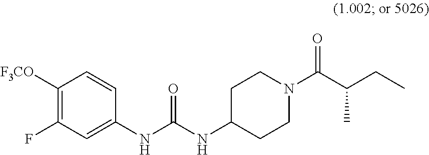 [SMILES: CC[C@H](C)C(=O)N1CCC(NC(=O)Nc2ccc(OC(F)(F)F)c(F)c2)CC1]

or a pharmaceutically acceptable salt thereof.

In some embodiments, the compound of Formula II has the structure

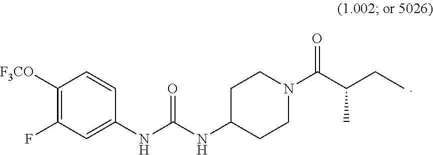 [SMILES: CC[C@H](C)C(=O)N1CCC(NC(=O)Nc2ccc(OC(F)(F)F)c(F)c2)CC1]

The compounds of Formula II may exist as salts. The present invention includes such salts. Typically, the salts used are pharmaceutically acceptable salts.

Pharmaceutically acceptable salts include salts of the active compounds which are prepared with relatively nontoxic acids or bases, depending on the particular substituents found on the compounds described herein. When compounds of Formula II contain relatively acidic functionalities, base addition salts can be obtained by contacting the neutral form of such compounds with a sufficient amount of the desired base, cither neat or in a suitable inert solvent. Examples of pharmaceutically acceptable base addition salts include sodium, potassium, calcium, ammonium, organic amino, or magnesium salt, or a similar salt. When compounds of Formula II contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of Formula II contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Certain compounds of Formula II can exist in unsolvated forms as well as solvated forms, including hydrated forms. In general, the solvated forms are equivalent to unsolvated forms and are encompassed within the scope of the present invention. Certain compounds of Formula II may exist in multiple crystalline or amorphous forms. In general, all physical forms are equivalent for the uses contemplated by the present invention and are intended to be within the scope of the present invention.

Certain compounds of Formula II possess asymmetric carbon atoms (optical centers) or double bonds; the enantiomers, racemates, diastereomers, tautomers, geometric isomers, stereoisomeric forms that may be defined, in terms of absolute stereochemistry, as (R)- or (S)- or, as (D)- or (L)- for amino acids, and individual isomers are encompassed within the scope of the present invention. The compounds of Formula II do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques.

Isomers include compounds having the same number and kind of atoms, and hence the same molecular weight, but differing in respect to the structural arrangement or configuration of the atoms.

It will be apparent to one skilled in the art that certain compounds of this invention may exist in tautomeric forms, all such tautomeric forms of the compounds being within the scope of the invention. Tautomer includes one of two or more structural isomers which exist in equilibrium and which are readily converted from one isomeric form to another.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, the compounds of Formula II may also contain unnatural proportions of atomic isotopes at one or more of the atoms that constitute such compounds. For example, the compounds of Formula II may be radiolabeled with radioactive isotopes, such as for example deuterium (²H), tritium (³H), iodine-125 (¹²⁵I), carbon-13 (¹³C), or carbon-14 (¹⁴C). All isotopic variations of the compounds of Formula II, whether radioactive or not, are encompassed within the scope of the present invention.

In addition to salt forms, the compounds of Formula II can be prepared as prodrugs. Prodrugs of the compounds described herein are those compounds that readily undergo chemical changes under physiological conditions to provide the compounds of Formula II. Additionally, prodrugs can be converted to the compounds of Formula II by chemical or biochemical methods in an ex vivo environment. For example, prodrugs can be slowly converted to the compounds of Formula II when placed in a transdermal patch reservoir with a suitable enzyme or chemical reagent.

The compounds of Formula II can be made by a variety of methods known in the art.

In some embodiments the sEH inhibitor is GSK2256294A

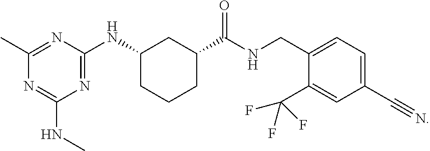 [SMILES: CNc1nc(C)nc(N[C@H]2CCC[C@@H](C(=O)NCc3ccc(C#N)cc3C(F)(F)F)C2)n1]

### Immunotherapeutic Agents

The methods disclosed herein include administering at least one immunotherapeutic agent. In some embodiments, one immunotherapeutic agent is administered. In some embodiments, two immunotherapeutic agents are administered. In some embodiments, one or two immunotherapeutic agents are administered. In some embodiments, three immunotherapeutic agents are administered.

The immunotherapeutic agents of the present disclosure include immune checkpoint inhibitors. A number of immune checkpoint inhibitors are known in the art and include both small molecule and antibodies, each of which are within the scope of the present disclosure.

Known immune checkpoint inhibitors include agents that block the activity of at least one of PD-1, PD-L1, BTLA, LAG-3, TIM-3, TIGIT or CTLA-4. These inhibitors can stimulate a T cell response in the subject with a cancer.

In some embodiments, the immune checkpoint inhibitor(s) block(s) the activity of at least one of PD-1, PD-L1, LAG-3, or CTLA-4.

In some embodiments, the immune checkpoint inhibitor(s) block(s) the activity of at least one of PD-1, PD-L1, or CTLA-4.

In some embodiments, two immune checkpoint inhibitors are administered: a first immune checkpoint inhibitor blocking the activity of PD-1 or PD-L1, and a second immune checkpoint inhibitor blocking the activity of LAG-3 or CTLA-4.

In some embodiments, two immune checkpoint inhibitors are administered: a first immune checkpoint inhibitor blocking the activity of PD-1 or PD-L1, and a second immune checkpoint inhibitor blocking the activity of LAG-3. In some embodiments, the first immune checkpoint inhibitor blocking the activity of PD-1 is nivolumab and the second immune checkpoint inhibitor blocking the activity of LAG-3 is relatlimab. In some embodiments, these two antibodies are administered in a fixed-dose combination (OPDUALAG™).

In some embodiments, two immune checkpoint inhibitors are administered: a first immune checkpoint inhibitor blocking the activity of PD-1 or PD-L1, and a second immune checkpoint inhibitor blocking the activity of CTLA-4.

In some embodiments, two immune checkpoint inhibitors are administered: a first immune checkpoint inhibitor blocking the activity of CTLA-4, and a second immune checkpoint inhibitor blocking the activity of LAG-3.

In some embodiments, one immune checkpoint inhibitor is administered, blocking the activity of PD-1 or PD-L1.

In some embodiments, one immune checkpoint inhibitor is administered, blocking the activity of PD-1.

In some embodiments, one immune checkpoint inhibitor is administered, blocking the activity of LAG-3.

In some embodiments, one immune checkpoint inhibitor is administered, blocking the activity of CTLA-4.

In some embodiments, the immune checkpoint inhibitor blocking the activity of PD-L1 is avelumab (BAVENCIO®), atezolizumab (TECENTRIQ®), or durvalumab (IMFINZI®).

In some embodiments, the immune checkpoint inhibitor blocking the activity of PD-L1 is avelumab (BAVENCIO®). In some embodiments, the immune checkpoint inhibitor blocking the activity of PD-L1 is atezolizumab (TECENTRIQ®). In some embodiments, the immune checkpoint inhibitor blocking the activity of PD-L1 is durvalumab (IMFINZI®).

In some embodiments, the immune checkpoint inhibitor blocking the activity of PD-1 is nivolumab (OPDIVO®), pembrolizumab (KEYTRUDA®), cemiplimab (LIBTAYO®), dostarlimab (JEMPERLI®), or zimberelimab.

In some embodiments, the immune checkpoint inhibitor blocking the activity of PD-1 is nivolumab (OPDIVO®). In some embodiments, the immune checkpoint inhibitor blocking the activity of PD-1 is pembrolizumab (KEYTRUDA®). In some embodiments, the immune checkpoint inhibitor blocking the activity of PD-1 is cemiplimab (LIBTAYO®). In some embodiments, the immune checkpoint inhibitor blocking the activity of PD-1 is dostarlimab (JEMPERLI®). In some embodiments, the immune checkpoint inhibitor blocking the activity of PD-1 is zimberelimab.

In some embodiments, the immune checkpoint inhibitor blocking the activity of LAG-3 is relatlimab.

In some embodiments, the immune checkpoint inhibitor blocking the activity of CTLA-4 is ipilimumab (YERVOY®) or tremelimumab.

In some embodiments, the immune checkpoint inhibitor blocking the activity of CTLA-4 is ipilimumab (YERVOY®). In some embodiments, the immune checkpoint inhibitor blocking the activity of CTLA-4 is tremelimumab.

### Chemotherapeutic Agents

In some aspects, the methods described herein include also administering (iii) one or more chemotherapeutic agents.

Examples of chemotherapeutic agents include without limitation alkylating agent(s) (e.g., nitrogen mustards, nitrogen ureas, ethylenimines, methylmelamines, alkyl sulfonates, carmustine, triazenes), platinum-coordination complexes (e.g., cisplatin, carboplatin, oxaliplatin, nedaplatin, saraplatin, lobaplatin, heptaplatin, and mixtures thereof), anti-metabolite(s) (e.g., folic acid analogs (e.g., methotrexate), pyrimidine analogs (e.g., capecitabine, 5-fluorouracil, 5-fluorodeoxyuridine, 5-fluorodeoxyuridine monophosphate, cytosine arabinoside, 5-azacytidine, gemcitabine), purine analogs (e.g., mercaptopurine, thioguanine, azathioprine, pentostatin, erythrohydroxynonyladenine, fludarabine, cladribine)), plant alkaloid(s) and/or terpenoid(s), vinca alkaloid(s) (e.g., vincristine, vinblastine, vinorelbine, and vindesine), podophyllotoxin(s) (e.g., etoposide and teniposide), camptothecin(s) (e.g., irinotecan and topotecan), anthracycline(s), aromatase inhibitor(s), taxane(s) (e.g., paclitaxel, taxol and docetaxel), topoisomerase inhibitor(s) (e.g., (Type I inhibitors: camptothecins, including irinotecan and topotecan; Type II Inhibitors: amsacrine, etoposide, etoposide phosphate, and teniposide), antibiotic(s) (e.g., dactinomycin, daunorubicin, doxorubicin, idarubicin, epirubicin, bleomycins, mitomycin), hormone(s), differentiating agent(s), kinase inhibitor(s) (e.g., Bevacizumab, BIBW 2992, Cetuximab, Imatinib, Trastuzumab, Gefitinib, Ranibizumab, Pegaptanib, Sorafenib, Dasatinib, Sunitinib, Erlotinib, Nilotinib, Lapatinib, Panitumumab, Vandetanib, E7080, Pazopanib, Mubritinib and Fostamatinib) and antineoplastic agent(s) (e.g., (dactinomycin, doxorubicin, epirubicin, fludarabine and bleomycin). Any chemotherapeutic agent being used to treat the cancer of interest can be co-administered in a combination therapy regime with the agent that increases EETs (e.g., an inhibitor of sEH, an EET, an EpDPE, an EpETE, and mixtures thereof). Chemotherapeutic agents of use are known in the art and described in reference texts, e.g., Physicians' Desk Reference, 71st Ed., 2017, PDR Network or Brunton and Knollmann, Goodman & Gilman's The Pharmacological Basis of Therapeutics, 13th edition, 2017, McGraw-Hill Education/Medical.

In some embodiments, the chemotherapeutic agent includes administration of a platinum coordination complex. Platinum coordination complex chemotherapy agents include cisplatin, carboplatin, oxaliplatin, nedaplatin, saraplatin, lobaplatin, heptaplatin, and mixtures thereof. In some embodiments, the described combinations allow for reduced or subtherapeutic closing of the platinum coordination complex chemotherapeutic agent.

In some embodiments, the chemotherapeutic agent includes administration of a pyrimidine analog. Pyrimidine analog chemotherapy agents include gemcitabine, cytarabine (a.k.a., cytosine arabinoside), capecitabine, 5-fluorouracil, 5 fluorodeoxyuridine, 5-fluorodeoxyuridine monophosphate, 5 azacytidine, and mixtures thereof. In some embodiments, the described combinations allow for reduced or subtherapeutic dosing of the pyrimidine chemotherapeutic agent.

In some embodiments, the one or more chemotherapeutic agents are each independently selected from the group consisting of a platinum coordination complex and a nucleoside analog.

In some embodiments, two chemotherapeutic agents are administered: a platinum coordination complex and a nucleoside analog. In some embodiments, the platinum coordination complex is cisplatin and the nucleoside analogue is gemcitabine.

In some embodiments the subject has bladder cancer and is administered a combination of gemcitabine and cisplatin.

In some embodiments the subject has prostate cancer and is administered docetaxel.

In some embodiments, the subject has pancreatic cancer and is administered gemcitabine.

### High Omega-3, Omega-6 Diet

In some aspects, the methods described herein further include administering a high omega-3 or omega-6 diet to the subject. High omega-3 or omega-6 diets can be achieved in a variety of ways including through the selection of foods that are consumed (meal consumption) as well as by pharmaceutical supplementation (e.g., a liquid, tablet, capsule, etc.). All methods of achieving a high omega-3 or a high omega-6 diet are included in the administering step described herein. Some subjects will already be on a high omega-3 or omega-6 diet before treatment, other subject will alter their diet so that they consume a high omega-3 or omega-6 diet after discussions with medical professionals.

In some embodiments, the subject is administered a high omega-3 diet. In some embodiments, the high omega-3 diet comprises one or more components selected from the group consisting of fish oil, salmon, mackerel, tuna, herring, sardines, arctic char, trout, eggs, flaxseeds, and flaxseed oil.

In some embodiments, the subject is administered a high omega-6 diet. In some embodiments, the high omega-6 diet comprises one or more components selected from the group consisting of soybeans, corn, safflower, safflower oils, nuts, seeds, meat, poultry, and eggs.

In some aspects, provided herein are methods of preventing, suppressing, or treating cancer in a subject or prolonging survival of a subject comprising administering to the subject a high omega-3 diet or a high omega-6 diet and at least one immunotherapeutic agent.

In some embodiments, the subject is administered a high omega-3 diet. In some embodiments, the high omega-3 diet comprises one or more components selected from the group consisting of fish oil, salmon, mackerel, tuna, herring, sardines, arctic char, trout, eggs, flaxseeds, and flaxseed oil.

In some embodiments, the subject is administered a high omega-6 diet. In some embodiments, the high omega-6 diet comprises one or more components selected from the group consisting of soybeans, corn, safflower, safflower oils, nuts, seeds, meat, poultry, and eggs.

In some embodiments, a high omega-3 diet or a high omega-6 diet comprise the subject consuming about 100 mg to 2,000 mg of omega-3 fatty acids or omega-6 fatty acids per day. In some embodiments, a high omega-3 diet or a high omega-6 diet comprise the subject consuming at least about 100 mg of omega-3 fatty acids or omega-6 fatty acids per day. In some embodiments, a high omega-3 diet or a high omega-6 diet comprise the subject consuming at least about 200 mg of omega-3 fatty acids or omega-6 fatty acids per day. In some embodiments, a high omega-3 diet or a high omega-6 diet comprise the subject consuming at least about 300 mg of omega-3 fatty acids or omega-6 fatty acids per day. In some embodiments, a high omega-3 diet or a high omega-6 diet comprise the subject consuming at least about 400 mg of omega-3 fatty acids or omega-6 fatty acids per day. In some embodiments, a high omega-3 diet or a high omega-6 diet comprise the subject consuming at least about 500 mg of omega-3 fatty acids or omega-6 fatty acids per day. In some embodiments, a high omega-3 diet or a high omega-6 diet comprise the subject consuming at least about 600 mg of omega-3 fatty acids or omega-6 fatty acids per day. In some embodiments, a high omega-3 diet or a high omega-6 diet comprise the subject consuming at least about 700 mg of omega-3 fatty acids or omega-6 fatty acids per day. In some embodiments, a high omega-3 diet or a high omega-6 diet comprise the subject consuming at least about 800 mg of omega-3 fatty acids or omega-6 fatty acids per day. In some embodiments, a high omega-3 diet or a high omega-6 diet comprise the subject consuming at least about 900 mg of omega-3 fatty acids or omega-6 fatty acids per day. In some embodiments, a high omega-3 diet or a high omega-6 diet comprise the subject consuming at least about 1,000 mg of omega-3 fatty acids or omega-6 fatty acids per day. In some embodiments, a high omega-3 diet or a high omega-6 diet comprise the subject consuming at least about 1,250 mg of omega-3 fatty acids or omega-6 fatty acids per day. In some embodiments, a high omega-3 diet or a high omega-6 diet comprise the subject consuming at least about 1,500 mg of omega-3 fatty acids or omega-6 fatty acids per day. In some embodiments, a high omega-3 diet or a high omega-6 diet comprise the subject consuming at least about 1,750 mg of omega-3 fatty acids or omega-6 fatty acids per day.

In some embodiments, subjects having a high omega-3 diet or a high omega-6 diet have at least one meal a day where the amount of omega-3 fatty acids or omega-6 fatty acids meet the amounts listed above. In some embodiments, subjects having a high omega-3 diet or a high omega-6 diet have at least two meals a day where the amount of omega-3 fatty acids or omega-6 fatty acids meet the amounts listed above. In some embodiments, subjects having a high omega-3 diet or a high omega-6 diet have at least three meals a day where the amount of omega-3 fatty acids or omega-6 fatty acids meet the amounts listed above.

In some embodiments the immunotherapeutic agent is an immune checkpoint inhibitor block the activity of at least one of PD-1, PD-L1, or CTLA-4. In some embodiments, one immune checkpoint inhibitor is administered, blocking the activity of PD-1.

### Particular Combinations

In some embodiments, a subject in need of cancer treatment is administered (i) a high omega-3 fatty acid diet and (ii) at least one immunotherapeutic agent. In some embodiments, the at least one immunotherapeutic agent is a PD-1 or a PD-L1 inhibitor. In some embodiments, the PD-1 inhibitor is nivolumab (OPDIVO®), pembrolizumab (KEYTRUDA®), cemiplimab (LIBTAYO®), dostarlimab (JEMPERLI®), or zimberelimab. In some embodiments, PD-L1 inhibitor is avelumab (BAVENCIO®), atezolizumab (TECENTRIQ®), or durvalumab (IMFINZI®).

In some embodiments, a subject in need of cancer treatment is administered (i) at least one immunotherapeutic agent, and (ii) Compound 1.045. In some embodiments, the at least one immunotherapeutic agent is a CTLA-4 inhibitor. In some embodiments, the CTLA-4 inhibitor is ipilimumab (YERVOY®) or tremelimumab.

In some embodiments, a subject in need of cancer treatment is administered (i) at least one immunotherapeutic agent, and (ii) Compound 1.002. In some embodiments, the at least one immunotherapeutic agent is a PD-1 or a PD-L1 inhibitor. In some embodiments, the PD-1 inhibitor is nivolumab (OPDIVO®), pembrolizumab (KEYTRUDA®), cemiplimab (LIBTAYO®), dostarlimab (JEMPERLI®), or zimberelimab. In some embodiments, PD-L1 inhibitor is avelumab (BAVENCIO®), atezolizumab (TECENTRIQ®), or durvalumab (IMFINZI®).

In some embodiments, a subject in need of cancer treatment is administered (i) at least one immunotherapeutic agent, and (ii) Compound 2.017. In some embodiments, the at least one immunotherapeutic agent comprises two immune checkpoint inhibitors: a first immune checkpoint inhibitor blocking the activity of PD-1 or PD-L1, and a second immune checkpoint inhibitor blocking the activity of CTLA-4. In some embodiments, the PD-1 inhibitor is nivolumab (OPDIVO®), pembrolizumab (KEYTRUDA®), cemiplimab (LIBTAYO®), dostarlimab (JEMPERLI®), or zimberelimab. In some embodiments, PD-L1 inhibitor is avelumab (BAVENCIO®), atezolizumab (TECENTRIQ®), or durvalumab (IMFINZI®). In some embodiments, the CTLA-4 inhibitor is ipilimumab (YERVOY®) or tremelimumab.

### Formulation and Administration

The agents for administration in the present methods (e.g., (i) at least one immunotherapeutic agent, (ii) a dual inhibitor of COX-2 and sEH, or (ii) an sEH inhibitor, sometimes further includes (iii) one or more chemotherapeutic agents) can be formulated and administered together (e.g., as a mixture) or separately. In some embodiments, the agents are administered via the same or different routes of administration. In some embodiments, the agents are co-administered concurrently or sequentially. The different pharmacologically active agents can be prepared and administered in a wide variety of oral, parenteral and topical dosage forms. In some embodiments, e.g., depending on the cancer being treated and other considerations, the agents can be administered together or separately orally, by injection, that is, intravenously, intramuscularly, intracutaneously, subcutaneously, intraduodenally, intravesically, intraperitoneally or intratumorally. The one or more agents can also be administered by inhalation, for example, intranasally or intrapulmonarily. Additionally, the one or more agents can be administered transdermally. Accordingly, in some embodiments, the one or more active agents are co-administered formulated in a pharmaceutically acceptable carrier or excipient.

For preparing the pharmaceutical compositions, the pharmaceutically acceptable carriers can be cither solid or liquid. Solid form preparations include powders, tablets, pills, capsules, cachets, suppositories, and dispersible granules. A solid carrier can be one or more substances which may also act as diluents, flavoring agents, binders, preservatives, tablet disintegrating agents, or an encapsulating material.

In powders, the carrier is a finely divided solid which is in a mixture with the finely divided active component. In tablets, the active component is mixed with the carrier having the necessary binding properties in suitable proportions and compacted in the shape and size desired. The powders and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution. Transdermal administration can be performed using suitable carriers. If desired, apparatuses designed to facilitate transdermal delivery can be employed. Suitable carriers and apparatuses are well known in the art, as exemplified by U.S. Pat. Nos. 6,635,274, 6,623,457, 6,562,004, and 6,274,166.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active components in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

A variety of solid, semisolid and liquid vehicles have been known in the art for years for topical application of agents to the skin. Such vehicles include creams, lotions, gels, balms, oils, ointments and sprays. See, e.g., Provost C. “Transparent oil-water gels: a review,” Int J Cosmet Sci. 8:233-247 (1986), Katz and Poulsen, Concepts in biochemical pharmacology, part I. In: Brodie B B, Gilette J R, eds. Handbook of Experimental Pharmacology. Vol. 28. New York, NY: Springer; 107-174 (1971), and Hadgcraft, “Recent progress in the formulation of vehicles for topical applications,” Br J Dermatol., 81:386-389 (1972). A number of topical formulations of analgesics, including capsaicin (e.g., Capsin®), so-called “counter-irritants” (e.g., Icy-Hot®, substances such as menthol, oil of wintergreen, camphor, or eucalyptus oil compounds which, when applied to skin over an area presumably alter or off-set pain in joints or muscles served by the same nerves) and salicylates (e.g. BenGay®), are known and can be readily adapted for topical administration. It is presumed that the person of skill is familiar with these various vehicles and preparations and they need not be described in detail herein.

The one or more pharmacologically active agents can be mixed into such modalities (creams, lotions, gels, etc.) for topical administration. In general, the concentration of the agents provides a gradient which drives the agent into the skin. Standard ways of determining flux of drugs into the skin, as well as for modifying agents to speed or slow their delivery into the skin are well known in the art and taught, for example, in Osborne and Amann, eds., Topical Drug Delivery Formulations, Marcel Dekker, 1989. The use of dermal drug delivery agents in particular is taught in, for example, Ghosh et al., eds., Transdermal and Topical Drug Delivery Systems, CRC Press, (Boca Raton, FL, 1997).

In some embodiments, the one or more active agents are formulated in a cream. Typically, the cream comprises one or more hydrophobic lipids, with other agents to improve the “feel” of the cream or to provide other useful characteristics.

In other embodiments, the one or more active agents are formulated in a lotion. Typical lotions comprise, for example, water, mineral oil, petrolatum, sorbitol solution, stearic acid, lanolin, lanolin alcohol, cetyl alcohol, glyceryl stearate/PEG-100 stearate, triethanolamine, dimethicone, propylene glycol, microcrystalline wax, tri (PPG-3 myristyl ether) citrate, disodium EDTA, methylparaben, ethylparaben, propylparaben, xanthan gum, butylparaben, and methyldibromo glutaronitrile.

In some embodiments, the agent is, or agents are, in an oil, such as jojoba oil. In some embodiments, the agent is, or agents are, in an ointment, which may, for example, white petrolatum, hydrophilic petrolatum, anhydrous lanolin, hydrous lanolin, or polyethylene glycol. In some embodiments, the agent is, or agents are, in a spray, which typically comprise an alcohol and a propellant. If absorption through the skin needs to be enhanced, the spray may optionally contain, for example, isopropyl myristate.

Whatever the form in which the agents are administered (that is, whether by lotion, gel, spray, etc.), they are preferably administered at a dosage of about 0.01 mg to 10 mg per 10 cm2.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The term “unit dosage form”, as used in the specification, refers to physically discrete units suitable as unitary dosages for human subjects and animals, each unit containing a predetermined quantity of active material calculated to produce the desired pharmaceutical effect in association with the required pharmaceutical diluent, carrier or vehicle.

Because the one or more agents can function in a cooperative or synergistic manner with other agents in the currently described methods, a reduced dosage or sub-therapeutic amount relative to the dose or amount needed for an efficacious response if the active agents were administered alone may be appropriate. The dosage of the specific compounds depends on many factors that are well known to those skilled in the art. They include for example, the route of administration and the potency of the particular compound. An exemplary dose of a dual COX-2 and sEH inhibitor is from about 0.1 mg/kg to about 10 mg/kg, 25 mg/kg, 50 mg/kg, 100 mg/kg, 150 mg/kg, 200 mg/kg, 250 mg/kg, 300 mg/kg, 350 mg/kg, 400 mg/kg, or 500 mg/kg body weight of the mammal. Doses of immunotherapeutic agents and chemotherapeutic agents are known in the art, and can be found, e.g., in the published literature and in reference texts, e.g., the Physicians' Desk Reference, 71st Ed., 2017, PDR Network, or Brunton and Knollmann, Goodman & Gilman's The Pharmacological Basis of Therapeutics, 13th edition, 2017, McGraw-Hill Education/Medical).

Determination of an effective amount is well within the capability of those skilled in the art, especially in light of the detailed disclosure provided herein. Generally, an efficacious or effective amount is determined by first administering a low dose or small amount of an agent and then incrementally increasing the administered dose or dosages, adding a second or third agent as needed, until a desired effect of is observed in the treated subject with minimal or no toxic side effects. Applicable methods for determining an appropriate dose and dosing schedule for administration of a combination of the present invention are described, for example, in Goodman and Gilman's The Pharmacological Basis of Therapeutics, 13th Edition, 2017, supra; in a Physicians' Desk Reference (PDR), 71st Edition, 2017 (PDR Network); in Remington: The Science and Practice of Pharmacy, 21^{st} Ed., 2005, supra; and in Martindale: The Complete Drug Reference, Sweetman, 2005, London: Pharmaceutical Press., and in Martindale, Martindale: The Extra Pharmacopoeia, 31st Edition., 1996, Amer Pharmaceutical Assn, each of which are hereby incorporated herein by reference.

In some embodiments, the dual inhibitor of COX-2 and sEH or sEH inhibitor is administered orally. In some embodiments, the immunotherapeutic agent is administered orally or by injection. In some embodiments, the at least one immunotherapeutic agent is administered by injection. In some embodiments, the at least one immunotherapeutic agent is administered by injection. In some embodiments, the one or more chemotherapeutic agents are administered by injection. In some embodiments, the one or more chemotherapeutic agents are administered by injection.

### Kits

Further provided herein are kits. In varying embodiments, the kits comprise (i) at least one immunotherapeutic agent, and (ii) a dual inhibitor of COX-2 and sEH; or (i) at least one immunotherapeutic agent, and (ii) an sEH inhibitor. The identity of the immunotherapeutic agents, dual inhibitor of COX-2 and sEH, and the sEH inhibitor are further discussed in other sections of this application.

In some embodiments, the kits also include (iii) one or more chemotherapeutic agents described herein.

Some of the kits described herein include a label describing a method of administering the agents included in the kit.

A kit may contain a label or packaging insert including identifying information for the components therein and instructions for their use (e.g., dosing parameters, clinical pharmacology of the active ingredient(s), including mechanism of action, pharmacokinetics and pharmacodynamics, adverse effects, contraindications, etc.). Labels or inserts can include manufacturer information such as lot numbers and expiration dates. The label or packaging insert may be, e.g., integrated into the physical structure housing the components, contained separately within the physical structure, or affixed to a component of the kit (e.g., an ampule, tube or vial).

Labels or inserts can additionally include, or be incorporated into, a computer readable medium. In some embodiments, the actual instructions are not present in the kit, but means for obtaining the instructions from a remote source, e.g., via the internet, are provided.

### I. EXAMPLES

The following examples are provided to illustrate but not limit the claimed invention.

### Example 1: Administration of an Immunotherapy and a Dual COX-2/sEH Inhibitor

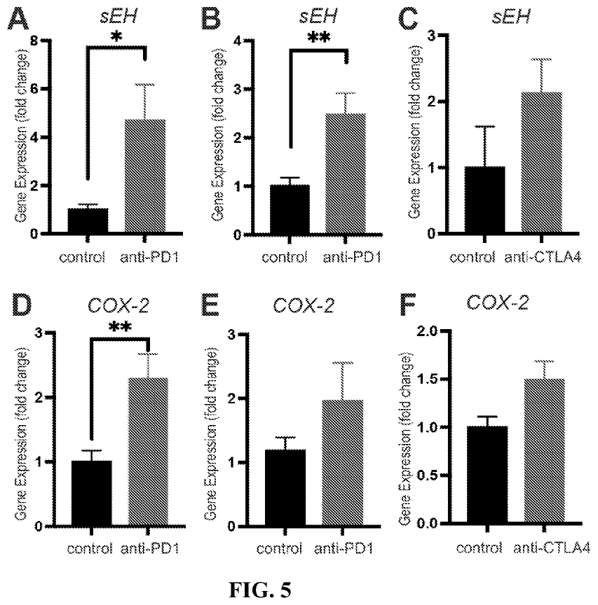

FIG. 5A-5F shows that immunotherapy can surprisingly induce sEH and COX-2 expression in cancer models. (A) Expression of sEH in RM1 prostate tumor tissue. Tumor cells (50,000 RM1 prostate cancer cells) were injected subcutaneously. Once tumors reached ˜200 mm³, treatment was initiated with either anti-PD1 (200 μg Q3D) or no treatment (control). Treatment with anti-PD1 increased gene expression of pro-inflammatory sEH. N=3-5 mice/group. p=0.0172. (B) Expression of sEH in MB49 bladder tumor tissue. Tumor cells (1×10⁶ MB49 bladder cancer cells) were injected subcutaneously. Once tumors reached ˜200 mm³, treatment was initiated with either anti-PD1 (200 μg Q3D) or no treatment (control). Treatment with anti-PD1 increased gene expression of pro-inflammatory sEH. N=3-5 mice/group. p=0.004. (C) Expression of sEH in MB49 bladder tumor tissue. Tumor cells (1×10⁶ MB49 bladder cancer cells) were injected subcutaneously. Once tumors reached ˜200 mm³, treatment was initiated with either anti-CTLA4 (200 μg 1^{st} dose, then 100 μg Q3D) or no treatment (control). Treatment with anti-CTLA4 led to a non-significant increase in gene expression of pro-inflammatory sEH. p=0.99. (D) Expression of COX-2 in RM1 prostate tumor tissue. Tumor cells (50,000 RM1 prostate cancer cells) were injected subcutaneously. Once tumors reached ˜200 mm³, treatment was initiated with either anti-PD1 (200 μg Q3D) or no treatment (control). Treatment with anti-PD1 increased gene expression of pro-inflammatory COX-2. N=3-5 mice/group. p=0.0081. (E) Expression of COX-2 in MB49 bladder tumor tissue. Tumor cells (1×10⁶ MB49 bladder cancer cells) were injected subcutaneously. Once tumors reached ˜200 mm³, treatment was initiated with either anti-PD1 (200 μg Q3D) or no treatment (control). Treatment with anti-PD1 led to a non-significant increase in gene expression of pro-inflammatory COX-2. N=3-5 mice/group. p=0.0825. (F) Expression of COX-2 in MB49 bladder tumor tissue. Tumor cells (1×10⁶ MB49 bladder cancer cells) were injected subcutaneously. Once tumors reached ˜200 mm³, treatment was initiated with either anti-CTLA4 (200 μg 1^{st} dose, then 100 μg Q3D) or no treatment (control). Treatment with anti-CTLA4 led to a non-significant increase in gene expression of pro-inflammatory COX-2. N=3-5 mice/group. p=0.9705.

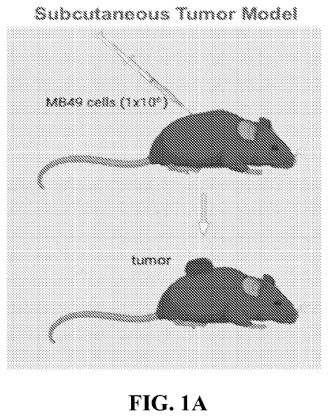

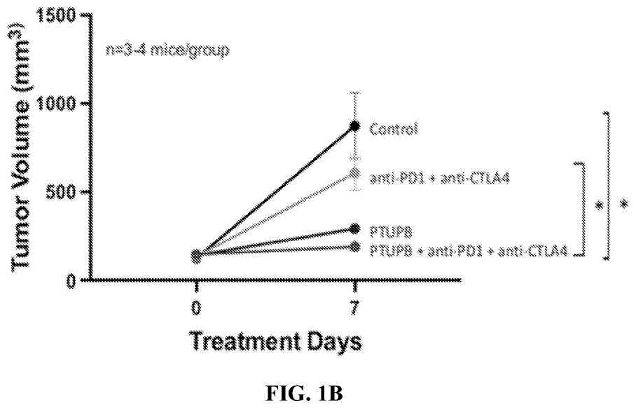

The combination of the a COX-2/sEH inhibitor (e.g. PTUPB (Compound 2.017)) and immunotherapy (e.g. anti-CTLA-4) provided an unexpected induction of tumor regression in an aggressive bladder cancer model. 10⁶ MB49 cells were introduced into mice, and treatment was initiated with established tumors (˜200 mm³), seven days after tumor injection. The CTLA-4 antibody used is from BioCell, Catalog number BE0131; In VivoMAb anti-mouse CTLA-4 (CD152). See, FIG. 1A and FIG. 1B.

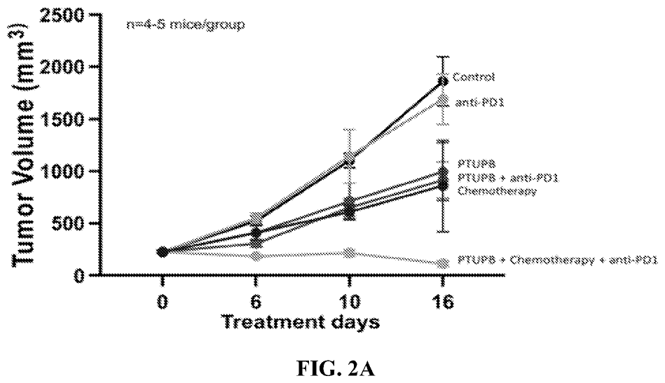

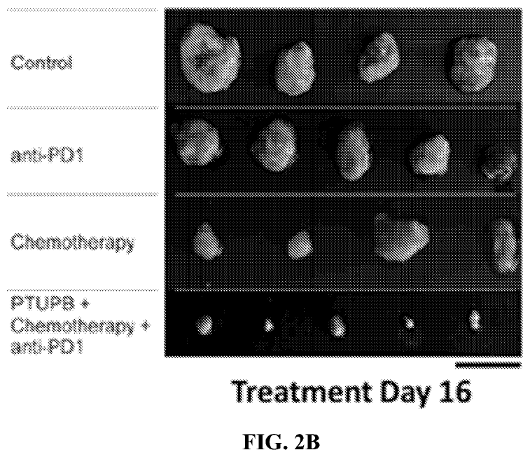

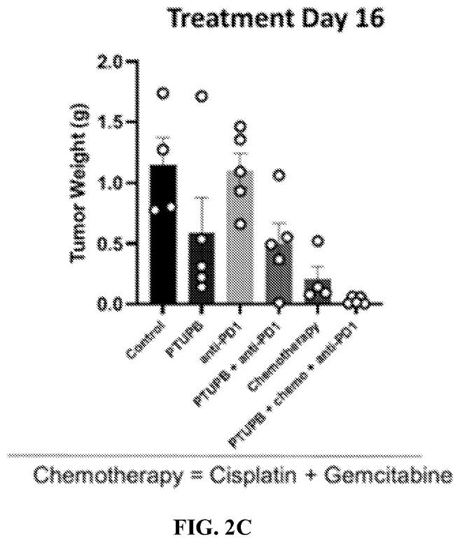

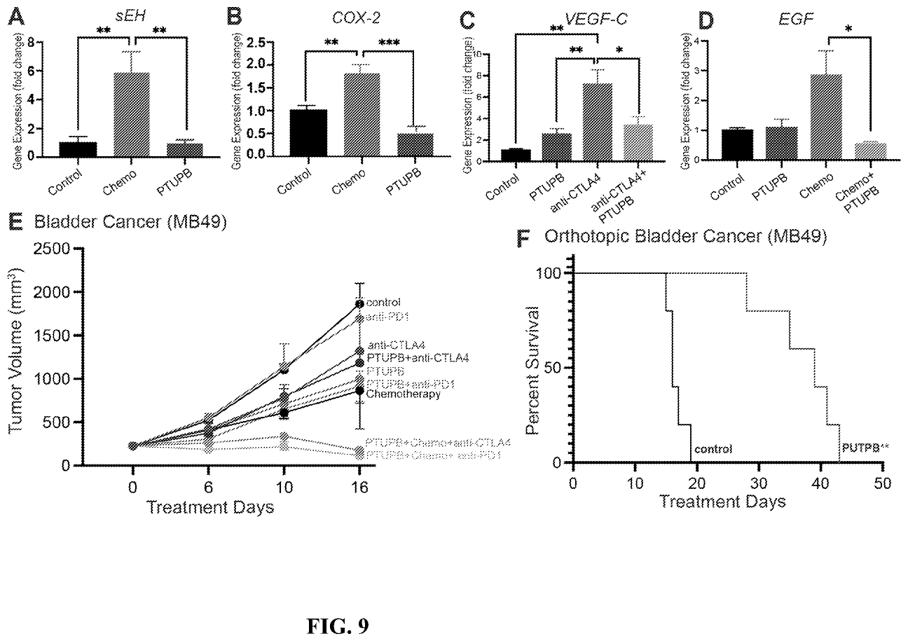

The combination of a dual COX-2/sEH inhibitor (e.g. PTUPB (Compound 2.017)), chemotherapy (e.g. gemcitabine and cisplatin), and immunotherapy (antibodies from BioXcell) provided an unexpected induction of tumor regression of bladder cancer. The mice in this study were randomized, and treatment was initiated with established tumors (˜200 mm³), seven days after treatment. See, FIG. 2A-2C; FIG. 9A-9F.

FIGS. 9A-F Dual COX-2/sEH inhibition blocks tumor growth and enhances chemotherapy and immunotherapy in murine bladder cancer model. (A & B). Mice were inoculated subcutaneously with 1×10⁶ MB49 cells. Treatment initiated when MB49 tumors reached ˜200 mm³. Mice were randomized and treated with PTUPB (60 mg/kg Q3D, orally) or chemotherapy (cisplatin+gemcitabine; cisplatin=5 mg/kg, gemcitabine=75 mg/kg, Q3D). Mice were sacrificed after 16 days of treatment. n=4-5 mice/group. Gene expression levels of sEH and COX-2 in tumor tissue after 16 days of treatment. Analyzed by qPCR and normalized by GAPDH. A: **Control vs chemotherapy (p=0.0099), **PTUPB vs chemotherapy (p=0.0095); B. **Control vs chemotherapy (p=0.0072), ***PTUPB vs chemotherapy (p=0.0001). (C) Mice were inoculated subcutaneously with 1×10⁶ MB49 cells. Treatment initiated when MB49 tumors reached ˜200 mm³. Mice were randomized and treated with combinations of PTUPB (60 mg/kg Q3D, orally), anti-PD1 (200 μg Q3D), anti-CTLA4 (100 μg first dose, 200 μg Q3D), chemotherapy (cisplatin+gemcitabine; cisplatin=5 mg/kg, gemcitabine=75 mg/kg, Q3D). Mice were sacrificed after 16 days of treatment. n=4-5 mice/group. *p<0.05. (D and E). Gene expression levels of EGF and VEGF-C in tumor tissue after 16 days of treatment. Analyzed by qPCR and normalized by GAPDH. D: *chemotherapy vs chemotherapy+PTUPB (p=0.0136); E: **Control vs anti-CTLA4 (p=0.0012), **PTUPB vs anti-CTLA4 (p=0.0081), **anti-CTLA4 vs anti-CTLA4+PTUPB (0.0296). (F) Mice were inoculated orthotopically into the bladder wall with 5×10⁵ MB49 cells. Treatment was initiated 3 days after tumor injection with PTUPB (60 mg/kg Q3D, orally). n=5 mice per group. **PTUPB vs control (p=0.0018).

### Example 2: Administration of an Immunotherapy and an sEH Inhibitor

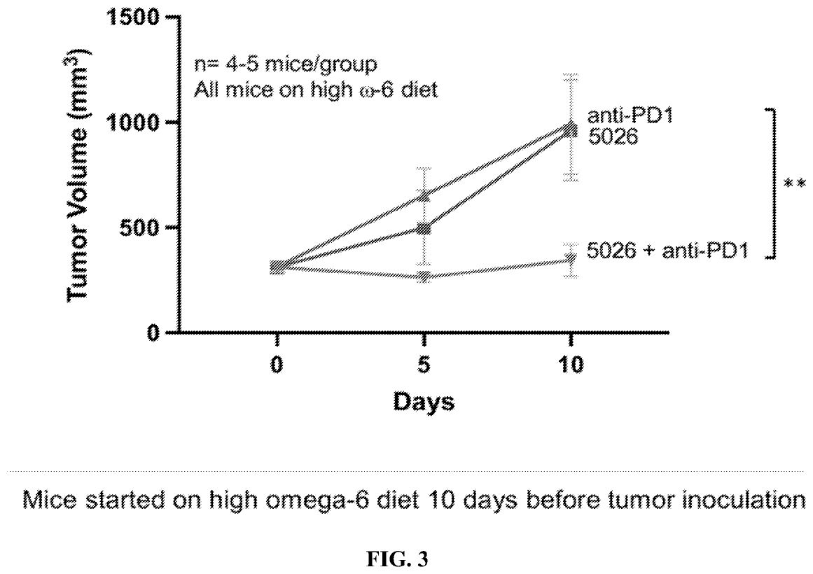

The combination of an sEH inhibitor (e.g. 5026 (Compound 1.002)) and immunotherapy (e.g. anti-PD-1) provided an unexpected induction of tumor regression in an aggressive bladder cancer model. See, FIG. 3.

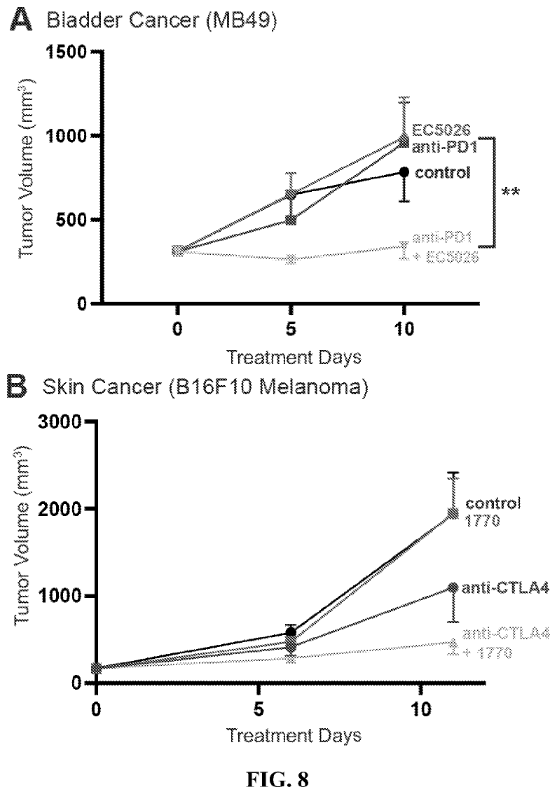

FIG. 8A and FIG. 8B also demonstrate unexpected induction of tumor regression in a bladder cancer model (8A) and a skin cancer model (8B) using a combination of an sEH inhibitor (e.g. 5026 (Compound 1.002)) and immunotherapy (e.g. anti-PD-1) (8A) and sEH inhibitor (e.g. 1770 (Compound 1.045)) and immunotherapy (e.g. anti-CTLA-4) (8B). (A) MB49 bladder cancer tumor volume over time. Following 12 days of pre-treatment with the omega-6 rich diet, tumor cells (1×10⁶ MB49 bladder cancer cells) were injected subcutaneously into the mid dorsum. Once tumors reached ˜200 mm³, treatment was initiated with anti-PD1 (200 μg Q3D), EC5026 (5 mg/kg/day via drinking water), combination anti-PD1+EC5026, or no treatment (control). Mice treated with EC5026+anti-PD1 had significantly smaller tumors than mice treated with EC5026 or anti-PD1 alone. N=3-5 mice per group. P<0.05. (B) B16F10 melanoma tumor volume over time. B16F10 melanoma tumor volume over time. Following 12 days of pre-treatment with the omega-6 rich diet, tumor cells (1×10⁶ B16F10 melanoma cells) were injected subcutaneously into the mid dorsum. Once tumors reached ˜200 mm³, treatment was initiated with anti-CTLA4 (200 μg 1^{st} dose, then 100 μg Q3D), 1770 (5 mg/kg/day via drinking water), combination 1770+anti-CTLA4, or no treatment (control). Mice treated with 1770+anti-CTLA4 tended to have smaller tumors than mice treated with 1770 or anti-CTLA4 alone. N=4-5 mice per group.

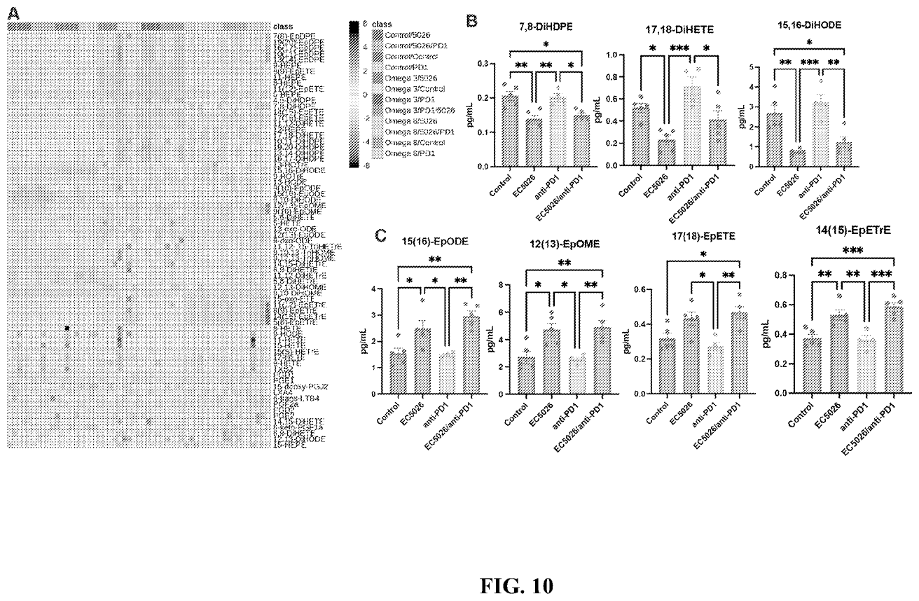

FIG. 10A-10C shows the altered oxylipin profiles in mice treated with omega-3 supplementation diets, immunotherapy, and sEH inhibitor. These lipid mediators are potential biomarkers of bladder cancer and the anti-tumor response to the diets (e.g. omega-3 or omega-6 fatty acids), immunotherapy (e.g., anti-PD1), and sEH inhibitor (e.g., 5026). (A) Heatmap of LCMS/MS oxylipin analysis performed on murine MB49 bladder cancer tumor tissue. (B) Treatment with anti-PD1 increased levels of 7,8-DiHDPE, 17,18-DiHETE, and 15,16-DiHODE in MB49 bladder cancer tissue. Treatment with EC5026 prevented this increase. Tumor tissue from mice treated with the control diet only. (C) Treatment with anti-PD1 decreased levels of 15(16)-EpODE, 12(13)-EpOME, 17(18)-EpETE and 14(15)-EpETrE in MB49 bladder cancer tissue. Treatment with EC5026 in combination with anti-PD1 prevented the drop in these metabolites, and increased levels of these metabolites compared to controls. Tumor tissue from mice treated with the control diet only.

### Example 3: Administration a High Omega-3 Diet

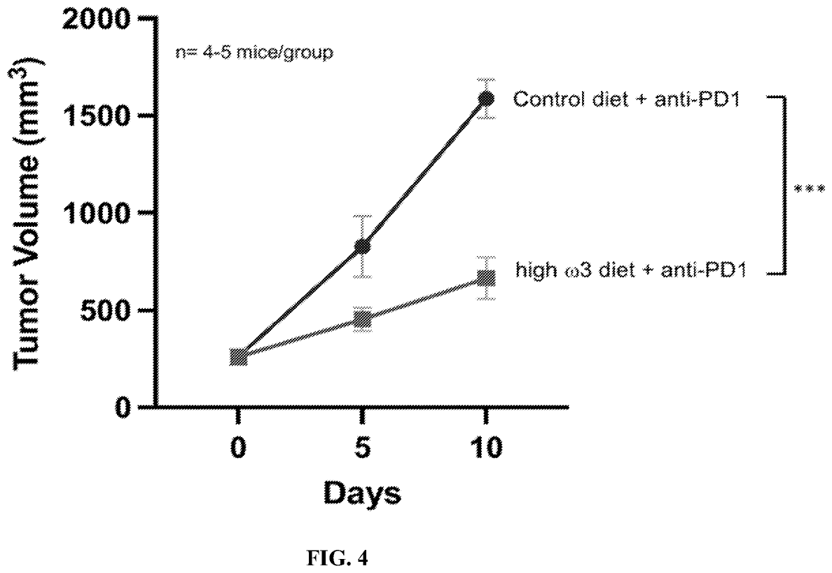

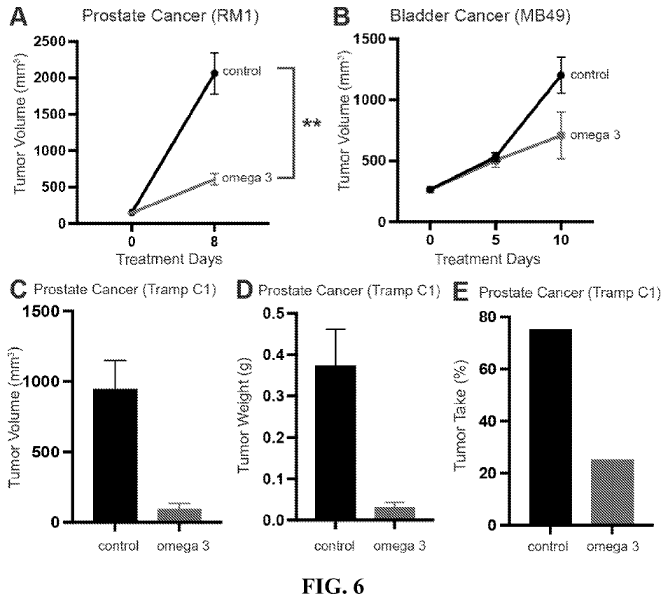

FIG. 4 and FIG. 6A-6E illustrate a surprising inhibition of tumor growth in both fast-growing and slow-growing tumor models in mice when the mice are fed dietary omega-3 supplementation.

FIGS. 6A-6E Dietary omega-3 supplementation inhibits tumor growth in both fast-growing and slow-growing tumor models in mice. (A) Prostate cancer (RM1) tumor growth over time. Following 12 days of pre-treatment with the omega-3 rich diet or control diet, tumor cells (1×10⁵ RM1 prostate cancer cells) were injected subcutaneously into the mid dorsum. Mice on the omega-3 rich diet had significantly smaller tumors than mice on the control diet (p=0.0009). N=3-5 mice/group. (B) MB49 bladder cancer tumor volume over time. Following 12 days of pre-treatment with the omega-3 rich diet or control diet, tumor cells (1×10⁶ MB49 bladder cancer cells) were injected subcutaneously. Mice on the omega-3 rich diet tended to have smaller tumors than mice on the control diet (p=0.0530). N=3-5 mice/group. (C & D) Prostate cancer (Tramp C1) tumor volume (C) tumor weight (D) at sacrifice. Following 12 days of pre-treatment with the omega-3 rich diet or control diet, tumor cells (1×10⁶ Tramp C1 prostate cancer cells) were injected subcutaneously. Mice on the omega-3 rich diet had significantly smaller tumors than mice on the control diet (p=0.0019). N=20 mice per group. (E) Prostate cancer (Tramp C1) tumor take rate. Following 12 days of pre-treatment with the omega-3 rich diet or control diet, tumor cells (1×10⁶ Tramp C1 prostate cancer cells) were injected subcutaneously. Fewer mice on the omega-3 diet developed tumors than mice on the control diet (p=0.001). N=20 mice per group.

### Example 4: Administration of an Immunotherapy and a High Omega-3 Diet

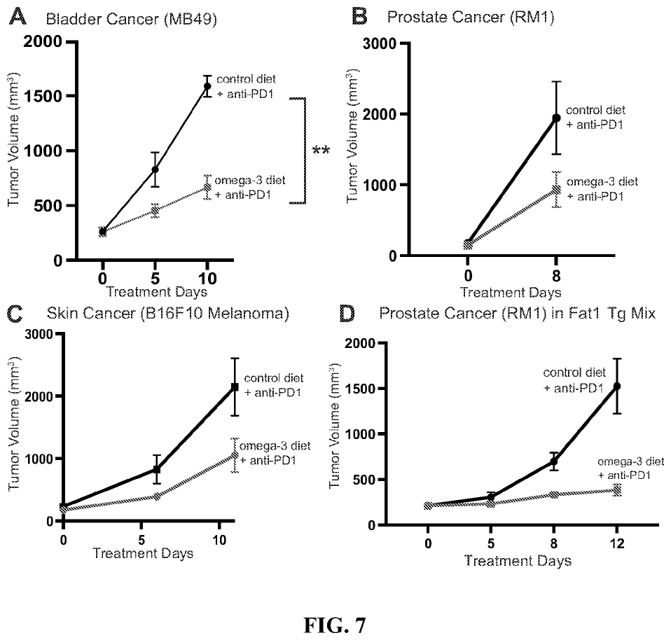

FIG. 7A-7D demonstrate an unexpected induction of tumor regression in a bladder cancer model (7A), a prostate cancer model (7B), a skin cancer model (7C), and a prostate cancer model (7D) when mice are administered immunotherapy and are fed dietary omega-3 supplementation. (A) MB49 bladder cancer tumor volume over time. Following 12 days of pre-treatment with the omega-3 rich diet or control diet, tumor cells (1×10⁶ MB49 bladder cancer cells) were injected subcutaneously into the mid dorsum. Once tumors reached ˜200 mm³, treatment was initiated with either anti-PD1 (200 μg Q3D) or no treatment (control). Mice on the omega-3 rich diet had significantly smaller tumors than mice on the control diet. N=3-5 mice per group. p<0.0001. (B) Prostate cancer (RM1) tumor volume over time. Following 12 days of pre-treatment with the omega-3 rich diet or control diet, tumor cells (1×10⁵ RM1 prostate cancer cells) were injected subcutaneously into the mid dorsum. Once tumors reached ˜200 mm³, treatment was initiated with either anti-PD1 (200 μg Q3D) or no treatment (control). Mice on the omega-3 rich diet tended to have smaller tumors than mice on the control diet N=3-5 mice per group. p=0.0835. (C) B16F10 melanoma tumor volume over time. Following 12 days of pre-treatment with the omega-3 rich diet or control diet, tumor cells (1×10⁶ B16F10 melanoma cells) were injected subcutaneously into the mid dorsum. Once tumors reached ˜200 mm³, treatment was initiated with either anti-CTLA4 (200 μg 1^{st} dose, then 100 μg Q3D) or no treatment (control). Mice on the omega-3 rich diet tended to have smaller tumors than mice on the control diet. N=3-5 mice per group. p=0.6599. (D) Prostate tumor growth in Fat-1 transgenic mice. Tumor cells (50,000 RM1 prostate cancer cells) were injected subcutaneously. Once tumors reached ˜200 mm³, treatment was initiated with either anti-PD1 (200 μg Q3D) or no treatment (control). Mice treated with anti-PD1 tended to have smaller tumors than untreated mice. N=3-5 mice/group. p=0.0558. As seen in these figures, dietary omega-3 supplementation enhances the anti-tumor activity of immunotherapy.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A method of preventing, suppressing, or treating cancer in a subject, the method comprising administering to the subject (i) at least one immunotherapeutic agent, and (ii) a dual inhibitor of COX-2 and sEH.

2. A method of prolonging survival of a subject in need of cancer treatment, the method comprising administering to the subject (i) at least one immunotherapeutic agent, and (ii) a dual inhibitor of COX-2 and sEH.

3. The method of claim 1 or 2, wherein the dual inhibitor of COX-2 and sEH is 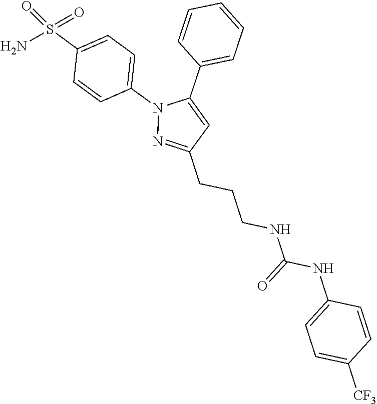 [SMILES: NS(=O)(=O)c1ccc(-n2nc(CCCNC(=O)Nc3ccc(C(F)(F)F)cc3)cc2-c2ccccc2)cc1]
or a pharmaceutically acceptable salt thereof.

4. A method of preventing, suppressing, or treating cancer in a subject, the method comprising administering to the subject (i) at least one immunotherapeutic agent, and (ii) an sEH inhibitor.

5. A method of prolonging survival of a subject in need of cancer treatment, the method comprising administering to the subject (i) at least one immunotherapeutic agent, and (ii) an sEH inhibitor.

6. The method of claim 4 or 5, wherein the sEH inhibitor is 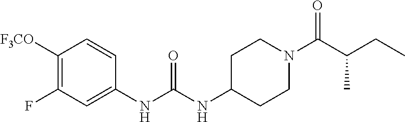 [SMILES: CC[C@H](C)C(=O)N1CCC(NC(=O)Nc2ccc(OC(F)(F)F)c(F)c2)CC1]
or a pharmaceutically acceptable salt thereof.

7. The method of any one of claims 1 to 5, wherein each immunotherapeutic agent is an immune checkpoint inhibitor that blocks the activity of at least one of PD-1, PD-L1, BTLA, LAG-3, TIM-3, TIGIT or CTLA-4.

8. The method of claim 7, wherein the immune checkpoint inhibitor(s) block(s) the activity of at least one of PD-1, PD-L1, LAG-3, or CTLA-4.

9. The method of claim 7, wherein the immune checkpoint inhibitor(s) block(s) the activity of at least one of PD-1, PD-L1, or CTLA-4.

10. The method of claim 7, wherein two immune checkpoint inhibitors are administered: a first immune checkpoint inhibitor blocking the activity of PD-1 or PD-L1, and a second immune checkpoint inhibitor blocking the activity of LAG-3 or CTLA-4.

11. The method of claim 7, wherein two immune checkpoint inhibitors are administered: a first immune checkpoint inhibitor blocking the activity of PD-1 or PD-L1, and a second immune checkpoint inhibitor blocking the activity of CTLA-4.

12. The method of claim 7, wherein one immune checkpoint inhibitor is administered, blocking the activity of PD-1 or PD-L1.

13. The method of claim 7, wherein one immune checkpoint inhibitor is administered, blocking the activity of PD-1.

14. The method of claim 7, wherein one immune checkpoint inhibitor is administered, blocking the activity of LAG-3.

15. The method of claim 7, wherein one immune checkpoint inhibitor is administered, blocking the activity of CTLA-4.

16. The method of any one of claims 7 to 12, wherein the immune checkpoint inhibitor blocking the activity of PD-L1 is selected from the group consisting of avelumab, atezolizumab, and durvalumab.

17. The method of any one of claims 7 to 13, wherein the immune checkpoint inhibitor blocking the activity of PD-1 is selected from the group consisting of nivolumab, pembrolizumab, cemiplimab, dostarlimab, and zimberelimab.

18. The method of any one of claim 7 to 11 or 15, wherein the immune checkpoint inhibitor blocking the activity of CTLA-4 is selected from the group consisting of ipilimumab and tremelimumab.

19. The method of any one of claims 1 to 18, further comprising administering (iii) one or more chemotherapeutic agents.

20. The method of claim 19, wherein the one or more chemotherapeutic agents are each independent selected from the group consisting of alkylating agent(s), platinum-coordination complexe(s), nucleoside analog(s), anti-metabolite(s), plant alkaloid(s) and/or terpenoid(s), vinca alkaloid(s), podophyllotoxin(s), camptothecin(s), anthracycline(s), aromatase inhibitor(s), taxane(s), topoisomerase inhibitor(s), antibiotic(s), hormone(s), differentiating agent(s), kinase inhibitor(s) and antineoplastic agent(s).

21. The method of claim 19, wherein the one or more chemotherapeutic agents are each independently selected from the group consisting of a platinum coordination complex and a nucleoside analog.

22. The method of claim 21, wherein two chemotherapeutic agents are administered: a platinum coordination complex and a nucleoside analog.

23. The method of claim 22, wherein the platinum coordination complex is cisplatin and the nucleoside analogue is gemcitabine.

24. The method of any one of claims 20 to 22, wherein the platinum coordination complex is selected from the group consisting of cisplatin, carboplatin, oxaliplatin, nedaplatin, saraplatin, lobaplatin, heptaplatin, and mixtures thereof.

25. The method of claim 24, wherein the platinum coordination complex is cisplatin.

26. The method of any one of claims 20 to 22, wherein the nucleoside analog is a pyrimidine analog, e.g., a deoxycytidine analog.

27. The method of claim 26, wherein the pyrimidine analog is selected from the group consisting of gemcitabine, cytarabine (a.k.a., cytosine arabinoside), capecitabine, 5-fluorouracil, 5-fluorodeoxyuridine, 5-fluorodeoxyuridine monophosphate, 5-azacytidine, and mixtures thereof.

28. The method of claim 27, wherein the nucleoside analogue is gemcitabine.

29. The method of any one of claims 1 to 28, further comprising administering (iv) a high omega-3 or a high omega-6 diet to the subject.

30. The method of any one of claims 1 to 29, wherein the subject has a cancer selected from the group consisting of: bladder, ovarian, cervical, breast, testicular, prostate, head and neck, oral, esophageal, gastric, lung, pancreatic, skin, leukemia, colon and colorectal cancer.

31. The method of any one of claim 30, wherein the subject has bladder cancer.

32. The method of any one of claims 1 to 31, wherein the subject is a mammal.

33. The method of claim 32, wherein the mammal is a human, a canine or a feline.

34. A method of preventing, suppressing, or treating cancer in a subject, the method comprising administering to the subject (i) a high omega-3 diet or a high omega-6 diet.

35. A method of prolonging survival of a subject in need of cancer treatment, the method comprising administering to the subject (i) a high omega-3 diet or a high omega-6 diet.

36. The method of claim 34 or claim 35, further comprising (ii) administering at least one immunotherapeutic agent.

37. The method of any one of claims 34 to 36, wherein the immunotherapeutic agent is selected from any one of claims 7 to 18.

38. The method of any one of claims 34 to 37, wherein the subject is administered (i) a high omega-3 diet.

39. The method of claim 38, wherein the high omega-3 diet comprises one or more components selected from the group consisting of fish oil, salmon, mackerel, tuna, herring, sardines, arctic char, trout, eggs, flaxseeds, and flaxseed oil.

40. The method of any one of claims 34 to 36 wherein the subject is administering (i) a high omega-6 diet.

41. The method of claim 40, wherein the high omega-6 diet comprises one or more components selected from the group consisting of soybeans, corn, safflower, safflower oils, nuts, seeds, meat, poultry, and eggs.
